(19) United States
(12) Patent Application Publication
(10) Pub. No.: US 2010/0145469 A1
(43) Pub. Date: Jun. 10, 2010

(54) BIOCERAMIC IMPLANTS HAVING BIOACTIVE SUBSTANCE

(76) Inventors: Jake Edward Barralet, Montreal (CA); Uwe Gbureck, Wurzburg (DE)

Correspondence Address: DOWELL & DOWELL P.C., 103 Oronoco St., Suite 220, Alexandria, VA, 22314, US

(21) Appl. No.: 12/526,223

(22) PCT Filed: Feb. 7, 2008
(86) PCT No.: PCT/CA08/00248

§ 371 Date: Feb. 16, 2010

Related U.S. Application Data
(60) Provisional application No. 60/888,708, filed on Feb. 7, 2007.

(51) Int. Cl. A61F 2/28; B05D 3/10

(52) U.S. Cl. 623/23.56; 427/2.24

(57) ABSTRACT

A bioceramic endoprosthesis includes a reservoir or deposition of a bioactive substance, such as an angiogenic growth factor, that can provide a biological function, such as vascularization of the endoprosthesis. Such a bioceramic can be prepared by a low temperature direct rapid prototyping inkjet printing system and process. Such a direct inkjet printing process includes the following: applying a ceramic powder to a substrate; inkjet printing a binder solution onto the ceramic powder so as to form a bound ceramic; inkjet printing a bioactive substance solution onto the bound ceramic, wherein the bioactive substance is printed on the bound ceramic at the low temperature (e.g., room temperature or within +/−10° C. of 25° C.); and repeating the process in order to form the bioceramic endoprosthesis.

### BACKGROUND OF THE INVENTION

1. The Field of the Invention

The present invention relates to bioceramic implants that include at least one bioactive substance. Additionally, the present invention relates to systems and processes for use in co-printing a bioceramic substrate and bioactive substances so as to produce bioceramic implants that include the bioactive substances. Furthermore, the present invention relates to systems and processes that employ inkjet printing technologies to produce bioceramic implants that can induce tissue repair, cell migration, cell proliferation, cell or tissue differentiation, wound healing, tissue growth, vascularization within and locally surrounding the bioceramic implant.

2. The Related Technology

Biocompatible materials are commonly used in the healthcare industry to provide various products for use in specific settings. Usually, the biocompatible material is a synthetic or natural material used to replace part of a living organism or to function in intimate contact with living tissue. While some biocompatible materials are configured to be used transiently within a living organism, other biocompatible materials are configured to be used as permanent implants. Also, some biocompatible materials that are used as implants are configured to operate as a substitute or replacement for an anatomical feature that is damaged, diseased, or nonfunctional, or within a healing compromised patient. However, even when the biocompatible implant is configured as a substitute or replacement for an anatomical feature, the implant material is usually different from the natural material produced by the living organism. For example, a biocompatible implant material, such as hydroxyapatite, configured to be a bone substitute may not have the features of a natural bone even though hydroxyapatite has a composition and properties similar to natural bone.

Bioceramics, such as hydroxyapatite, have been used in orthopedic surgical settings as implants. Usually, the bioceramic is used as a biocompatible coating on another material, as a body of an implant, or as an endoprosthesis. Bioceramic coatings usually do not have adverse interactions with the tissue surrounding them and can protect the living organism from an underlying material. Bioactive osteoconductive calcium-phosphate coatings ensure the growth of bone tissue over its surface, and osteoconductive hydroxyapatite compositions ensure the formation of new bone on its surface. Additionally, a bioceramic endoprosthesis, such as a bone graft substitute, can be used for building up of bone and filling hollows of missing, diseased, non-functional, or damaged bone. However, it is preferred that bioceramic endoprostheses have the necessary porosity to provide the intergrowth of bone tissue into the artificial implant pores, and strength to withstand the implantation procedure and use before bone growth is complete.

It has been found that pores are important in a bioceramic endoprosthesis because they are conduits for blood supply and hence tissue growth. Pores can also provide a way for living bone to attach itself permanently to a bioceramic endoprosthesis. Also, pore geometry of a bioceramic endoprosthesis has been found to be an important factor in bone healing.^{[1-2]} Typically, it is preferred for the pore to be larger than 200 microns or even larger then 300 microns in diameter.

Bioceramic endoprostheses can be prepared into a variety of shapes and sizes using well-established processes for manufacturing ceramics. Recently, direct rapid prototyping processes have been used to prepare bioceramics in order to control the geometry and composition of a bioceramic endoprosthesis.^{[3-4]} However, current direct rapid prototyping processes include a high temperature sintering step.^{[5-7]} Such sintering (e.g., high temperature) can limit the types of materials that can be included in the bioceramic endoprosthesis. For example, the sintering step can preclude the ability to include organic compounds and bioactive substances within the bioceramic endoprosthesis, and the endoprosthesis itself cannot be made from thermally unstable ceramic compounds such as hydrated calcium phosphates.

On the other hand, current low temperature rapid prototyping methods are indirect, whereby slurries of calcium phosphate cement are impregnated into a negative pattern, such as in a wax material. After the cement sets, the negative wax pattern is dissolved at room temperature or melted to leave the desired pore geometry. Such low temperature rapid prototyping processing can allow for some bioactive substances to be included within the bioceramic endoprosthesis. However, these low temperature rapid prototyping processes are indirect and are not as efficient as direct rapid prototyping processes.

Accordingly, it would be advantageous to have a bioceramic endoprosthesis that includes a bioactive substance that can stimulate tissue repair, cell migration, cell proliferation, cell or tissue differentiation, would healing, tissue growth, induce vascularization within and locally surrounding the endoprosthesis. Additionally, it would be advantageous to have a direct rapid prototyping system and process to manufacture the bioceramic endoprosthesis. Furthermore, it would be advantageous to have a system and process that employs three-dimensional printing technologies in the direct rapid prototyping system and process to co-print the ceramic and bioactive substance to form the bioceramic endoprosthesis.

### SUMMARY OF THE INVENTION

In one embodiment, the present invention is a direct printing method for preparing a bioceramic endoprosthesis having a bioactive substance. Such a method can include the following: applying a ceramic powder to a substrate; spraying a binder solution onto the ceramic powder so as to form a bound ceramic; depositing at least one bioactive substance solution onto the bound ceramic so as to incorporate the bioactive substance with bound ceramic at a temperature that does not degrade the bioactive substance; and repeating.

In one embodiment, the present invention is a method for preparing a bioceramic endoprosthesis having a bioactive substance. Such a method can include the following: providing a ceramic powder; forming a ceramic endoprosthesis; and depositing at least one bioactive substance to the ceramic endoprosthesis so as to incorporate the bioactive substance with the ceramic endoprosthesis at a temperature that does not degrade the bioactive substance.

In one embodiment, the present invention is a direct printing method for preparing a bioceramic endoprosthesis. Such a method can include the following: applying a ceramic powder to a substrate; spraying a binder solution onto the ceramic powder so as to form a bound ceramic; depositing at least one hydrogel or polymer onto the bound ceramic so as to incorporate the hydrogel or polymer with bound ceramic; and repeating.

In one embodiment, the present invention is a method for preparing a bioceramic endoprosthesis. Such a method can include the following: providing a ceramic powder; forming a ceramic endoprosthesis; and depositing at least one hydrogel or polymer to the ceramic endoprosthesis so as to incorporate the hydrogel or polymer with the ceramic endoprosthesis.

In one embodiment, the method for forming the ceramic endoprosthesis includes at least one of rapid prototyping, molding, machining, or compacting.

In on embodiment, the method further includes applying a hardening solution to the ceramic, and hardening the ceramic into a hardened ceramic having the bioactive substance.

In one embodiment, the method further includes applying an aqueous solution to the hardened ceramic, and maintaining a hydrothermal-conversion temperature of the hardened ceramic while in contact with the aqueous solution so as to further harden the hardened ceramic, wherein the hydrothermal-conversion temperature is higher than the low temperature.

In one embodiment, the bioactive substance is not homogeneously distributed in the endoprosthesis.

In one embodiment, the bioactive substance is deposited in discrete locations in the endoprosthesis.

In one embodiment, the bioactive substance is selected from the group consisting of extracellular matrix component, synthetic extracellular matrix component, proteins, peptides, polypeptides, drugs, cytokines, DNA, RNA, cells, bone-inducing factors, bone morphogenic proteins (BMPs), growth factors, extra cellular matrix proteins (ECM), epidermal growth factor-growth factor family (EGF), transforming growth factor alpha or beta (TGF alpha, TGF beta), hepatocyte growth factor (HGF/SF), heparin-binding epidermal growth factor (EGF), basic fibroblast growth factor (bFGF), acidic fibroblast growth factor (aFGF), other fibroblast growth factors (FGF), keratinocyte growth factor (KGF), transforming growth factors (TGF) (e.g., beta-1, beta-2, and beta-3), platelet derived growth factor (PDGF), vascular endothelial growth factors (VEGF), tumor necrosis factor (TNF), interleukin-1 (IL-1), interleukin-6 (IL-6), other interleukin/cytokine family members, insulin-like growth factor 1 (IGF-1), colony-stimulating factor 1 (CSF-1), and granulocyte macrophage colony stimulating factor (GM-CSF), copper, copper salt, copper amino acid chelate, copper sulfate, selenium, selenium salt, selenium amino acid chelate, cobalt, cobalt salt, cobalt amino acid chelate, mammalian cell, a transformed mammalian cell configured to produce a bioactive substance, and combinations thereof.

In one embodiment, the mammalian cell or transformed mammalian cell is characterized by at least one of the following: being combined with a hydrogel carrier; part of a heterogenous population of cells types; includes platelet rich plasma (PRP); are autologous cells; are allogeneic cells; have been lethally irradiated; or have been treated exogenously with a growth factor.

In one embodiment, the ceramic powder is selected from the group consisting of bioinert ceramic, alumina, surface-bioactive ceramics, silicon carbide, zirconia, hydroxyapatite (HA), bioglasses, resorbable bioactive ceramics, alpha and/or beta tricalcium phosphates (TCP), tetracalcium phosphate (TTCP), octacalcium phosphate, calcium sulfate, dicalcium phosphate dihydrate (DCPD), hydrated calcium phosphates, calcium hydrogen phosphate, dicalcium phosphate anhydrous (DCPA), low-crystallinity HA, calcium pyrophosphates (anhydrous or hydrated), calcium polyphosphates (n≧3), calcium polyphosphate, calcium silicates, calcium carbonate, amorphous calcium salts, whitlockite, zeolite, artificial apatite, brushite, calcite, gypsum, phosphate calcium ore, iron oxides, calcium sulphate, magnesium phosphate, calcium deficient apatites, amorphous calcium phosphates, combinations thereof, crystalline forms thereof, amorphous forms thereof, anhydrous forms thereof, or hydrated forms thereof.

In one embodiment, the methods of the present invention can further include the following: fabricating the bioceramic endoprosthesis so as to have at least one pore having a diameter greater than about 200 microns; and localizing a portion of the bioactive substance within a ceramic matrix adjacent to or on a surface of the pore.

In one embodiment, the methods of the present invention can further include fabricating the bioceramic endoprosthesis so as to have at least one longitudinal channel, pore, wedge, groove, slot, corrugation, or spoke extending through the endoprosthesis so as to direct tissue growth therethrough.

In one embodiment, the methods of the present invention can further include combining a pharmacologic excipient with the endoprosthesis. Such excipient can be suitable for orthopedics. Examples of excipients can be those well known in the art as well as fibrin, fibrin sheets, and cell stabilizing composites.

In one embodiment, the present invention is a bioceramic endoprosthesis prepared by a method of the invention as described herein. Such a bioceramic endoprosthesis can include a biocompatible ceramic matrix having a body defining a external surface of the endoprosthesis, and at least one of a bioactive substance, hydrogel, or polymer. The bioactive substance, hydrogel, or polymer can be spatially localized within the endoprosthesis.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1A is a schematic diagram illustrating an embodiment of the system and process for direct rapid prototyping 3D printing of ceramic and bioactive substance to form a bioceramic having reservoirs of bioactive substance.

FIG. 1B is a schematic diagram illustrating an embodiment of the system and process for direct rapid prototyping 3D printing of HA and DCPD at low temperature to form the bioceramic, and shows the phase composition, compressive strength (CS), relative porosity, and specific surface area (SSA) during the printing, hardening, and hydrolysis stages.

FIG. 1C includes scanning electron micrographs showed the set cement microstructures to consist of 10-20 μm angular particles of unreacted cement components in a matrix of 5-10 μm tabular crystals of DCPD or 2-5 μm plate-like crystals of HA.

FIG. 2A is a schematic diagram illustrating an embodiment of mating halves of a DCPD implant (8×8×3 mm) with Y shaped macropore in the x-y plane before implantation. The main pore opening is marked 1. The ranched pore opening is marked 2. The closed pore end is marked 3.

FIG. 2B is a schematic diagram illustrating an embodiment of the assembled implant illustrating the orientation of the image in FIG. 2C.

FIG. 2C is a schematic diagram illustrating an embodiment of a superimposed reconstruction of three Y shaped pores (different shades) showing dimensional tolerance achieved (±110 μm) from μCT data. Dots around the pores are micropores in the cement matrix.

FIG. 2D is a schematic diagram illustrating an embodiment of the superimposed μCT images of FIG. 2C at a slice 100 μm below the centerline through three assembled cuboids. Sharp corners are less well reproduced than curved and straight features.

FIG. 3A are photographs of DCPD implants with (i) one untreated and (ii) one treated with VEGF at the blind end of the closed pore. Tissue in-growth in control implants (untreated) extended about 2 mm into open pores. In contrast well formed vascular tissue extended from the main pore opening to closed pore end and, to a limited extent, along the open branch pore in factor treated implants. Microscopic observation (far right column), shows microvessel formation in the tissue only in the closed pore ends of the VEGF treated implants.

FIG. 3B are photographs of light microscopy of HPS stained paraffin sections of tissue found in or near the closed pore ends of the implants. In contrast to untreated implants (i), organized connective tissue (arrowheads) was observed in the angiogenic factor loaded implants (ii). Field width is 960 μm.

FIG. 3C is a bar graph depicting the mean distance (+s.d.) covered by microvessels relative to total distance (dashed line) from main open pore to closed pore end.

FIG. 4A is a schematic representation of a photograph of barium chloride loaded DCPD cylinder (35×20 mm). A curved central pore runs through the center.

FIG. 4B is a schematic representation of a rendered μCT reconstruction of FIG. 4A showing a barium chloride loaded DCPD cylinder (35×20 mm). A curved central pore is colored white and runs through the center. Radiopaque barium chloride treated regions are light grey. The DCPD cement is colored darker grey and surrounds the lighter grey radiopaque barium chloride regions.

FIG. 6A provides examples of complex 3D shapes made in DCPD, which shows a disc with 32×1.5 mm diameter holes, and human skulls made by reducing the scale of CT data by a factor of 4, and one skull is sectioned to show internal detail.

FIG. 6B is a schematic diagram of a 2D branched structure hand drawn using a computer aided design (CAD) package reproduced in a 25×25×5 mm HA cuboid.

FIG. 6C is an X-ray photograph of the 2D branched structure of FIG. 6B. The X-ray photograph confirms the 2D branched structure of the computer aided design (CAD) package reproduced in a 25×25×5 mm HA cuboid.

FIG. 7 depicts adsorption of concentration gradients of serum proteins and saline on DCPD that shows the implants were stable (i.e., gradients were stable) in vitro for up to 3 weeks. The photographs show the plan view and cross section. Field widths: plan view 6 mm, cross section 5 mm.

FIGS. 8A-8E are schematic representations illustrating embodiments of bioceramic endoprosthesis in accordance with the present invention.

### DETAILED DESCRIPTION OF THE PREFERRED EMBODIMENTS

Generally, embodiments of the present invention include a bioceramic endoprosthesis impregnated with a bioactive substance, such as angiogenic growth factor, mammalian cell, transformed mammalian cell, bacteria cell, or transformed bacteria cell, or the like, which can induce tissue repair, cell migration, cell proliferation, cell or tissue differentiation, would healing, tissue growth, induce vascularization within and locally surrounding the endoprosthesis. It should be understood that an endoprosthesis is an object that can be implanted into any part of a patient's body. For example, an endoprosthesis can be an implant that is implanted into an arm, leg, head, mouth, jaw, torso, and the like. As such, the terms endoprosthesis and implant are substantially synonymous and can be used interchangeably.

An endoprosthesis or implant in accordance with the present invention can also be a ceramic that is configured for oral administration. Accordingly, the endoprosthesis can be configured similarly to a pill. It can be beneficial for such an orally administered endoprosthesis to retain the bioactive agent while passing through the stomach so that it can be released in the intestines. Similarly, the endoprosthesis or implant in accordance with the present invention can also be a ceramic that is configured as a suppository.

The endoprosthesis can include any biocompatible ceramic that can be fabricated into an endoprosthesis for implantation into a living organism using the system and processes described herein. Additionally, the endoprosthesis can include any bioactive substance that is biologically active, such as those that can facilitate vascularization of the endoprosthesis or other biological function. Furthermore, embodiments of the present invention can include a system and process to prepare the bioceramic implant. Such a system and process can include a direct rapid prototyping printing system and process to manufacture the bioceramic implant. Also, the direct rapid prototyping system and process can use inkjet printing technologies to co-print the ceramic and bioactive substance to form the bioceramic implant. Moreover, the system and process can operate at low temperatures, such as room temperature, so that temperature-sensitive bioactive substance, such as angiogenic proteins or cells, can be integrated into the bioceramic.

I. Bioceramic

The bioceramic endoprosthesis can be configured to include features that can promote vascularization, tissue morphogenesis, and/or other biological function. As such, the bioceramic endoprosthesis can be configured to increase concentrations of cell signalling molecules in the vicinity of the endoprosthesis and within the pores of the endoprosthesis. In order to increase the concentrations of cell-signalling molecules and enhance signals associated with tissue growth and repair, the bioceramic can provide controlled release of a bioactive substance, such as bioactive ions and molecules, in three dimensions. Such release of bioactive substances can be useful for stimulating and guiding tissue regeneration.

The bioceramic endoprosthesis can be configured into various forms. As such, the endoprosthesis can be a bone substitute, bone grafts, substitute for autograft, implant, medical device, stent, coating, or the like. The bioceramic material can include the bioactive substance at distinct locations or distributions throughout the bioceramic matrix.

The bioceramic endoprosthesis can be configured to be a macroporous osteoconductive bioceramic that can be used for bone grafting. The pores can allow bone to grow into the endoprosthesis. As such, the endoprosthesis can have varying porosity in order to allow vasculature to form within the pores. Also, the pores can be large enough to allow vessels, which may have the same or different sizes, to form within the pores. For example, the endoprosthesis can include a relative porosity from about 40% to about 95% and include pores that are from about 200 microns to about 4000 microns. However, the endoprosthesis can be configured to have a higher or lower porosity depending on the use and the loads that may be applied after implantation. Further, the pores can be larger than 300 microns in order to allow formation of more vasculature or bone after implantation. The features of the bioceramic endoprosthesis are described in more detail below.

Also, the bioceramic endoprosthesis can include at least one longitudinal channel, pore, wedge, groove, slot, corrugation, or spoke extending through the endoprosthesis so as to direct tissue growth therethrough. When the bioceramic includes such features, it may or may not have pores. Also, when such features are present as primary channels or the like, the endoprosthesis can be devoid of a secondary network of channels or the like. Additionally, when the endoprosthesis includes a plurality of such features, the features may or may not be interconnected.

The bioactive substance can be fabricated into the bioceramic by being included in within the ceramic matrix, such as being impregnated within the matrix, disposed within lattice, disposed within the ceramic, disposed on a ceramic surface, disposed in spatial locations, disposed within depositions, disposed within solid or liquid reservoirs, or by being deposited at discrete locations. This can include the depositions or reservoirs being disposed within discrete locations within the bioceramic matrix, such as near an external surface, adjacent to a pore, at the end of a closed pore, circumferentially around a pore, or on a pore surface. Also, the depositions or reservoirs can be macro, micro, or nano reservoirs, which can include depositions or reservoirs consisting of one or more bioactive substances, wherein the reservoirs can be solid, liquid, gel, paste, or the like. In some instances, the bioactive substance can be deposited within the endoprosthesis so as to form a pore surface. The bioactive substance can be included regions that entrap the bioactive substance, incorporate the bioactive substance, absorb the bioactive substance, such as macro, micro, or nano pore surfaces. Also, a pore can be configured to be a bioactive substance depot or reservoir that forms into a pore as the bioactive substance diffuses or dissolves into the body of the living organism. In instances where the bioceramic endoprosthesis includes other anatomically similar features, the bioactive substance can be properly positioned with respect to such features in order for the bioceramic matrix to release the bioactive substance with respect to the features. Moreover, the bioactive substance can be homogeneously distributed throughout the bioceramic, or it can be distributed at varying concentrations or concentration gradients in two or three dimensions. Alternatively, the bioactive substance can be printed onto or within an internal or external surface of a bioceramic in accordance with the processes described herein.

Multiple bioactive substances may be disposed within the endoprosthesis so as to be capable of being released simultaneously and/or sequentially. The release of multiple bioactive substances can provide multiple tissue growth or repair signals. This can be performed by printing different bioactive substances at different depths from a pore or outer surface, or by co-printing the bioactive substance with polymers to control release of the bioactive substance.

The bioceramic can be comprised of ceramic materials that are hardened into a ceramic matrix. For example, the cement powder can be configured as a cementitious material that hydrates or cures into a hardened bioceramic. Examples of bioceramic materials can include bioinert ceramic, alumina, surface-bioactive ceramics, silicon carbide, zirconia, hydroxyapatite (HA), bioglasses, resorbable bioactive ceramics, alpha and/or beta tricalcium phosphates (TCP), tetracalcium phosphate (TTCP), octacalcium phosphate, calcium sulfate, dicalcium phosphate dihydrate (DCPD), hydrated calcium phosphates, calcium hydrogen phosphate, dicalcium phosphate anhydrous (DCPA), low-crystallinity HA, calcium pyrophosphates (anhydrous or hydrated), calcium polyphosphates (n≧3), calcium polyphosphate, calcium silicates, calcium carbonate, amorphous calcium salts, whitlockite, zeolites, artificial apatite, brushite, calcite, gypsum, phosphate calcium ore, iron oxides, calcium sulphate, magnesium phosphate, calcium deficient apatites, amorphous calcium phosphates, and combinations thereof. Various ceramics can be crystalline, amorphous, glassy, anhydrous, or hydrated. Ceramics generally contain one or more of titanium, zinc, aluminium, zirconium, magnesium, potassium, calcium, iron, ammonium and sodium ions or atoms in addition to one or more of an oxide, a phosphate (ortho, pyro, tri, tetra, penta, meta, poly etc), a silicate, a carbonate, a nitride, a carbide, a sulphate, ions thereof, or the like. Also, other materials with similar properties that can be fabricated into a ceramic as described herein can be included in the present invention.

The bioactive substance can be any biological or synthetic compound, element, or substance that can provide a biological function. For example, the bioactive substance can induce tissue repair, cell migration, cell proliferation, cell or tissue differentiation, would healing, tissue growth, induce vascularization within and locally surrounding the endoprosthesis. As such, the bioactive substance can be an extracellular matrix component, synthetic extracellular matrix component, an angiogenic factor, growth/cytokine factor, or a combination of angiogenic factor and growth/cytokine factor, drug, peptide, polypeptide, active peptide sequences, DNA, RNA, cells, bone-inducing factors (e.g., bone morphogenic proteins (BMPs)), growth factors, and the like. Examples of suitable bioactive substances include extra cellular matrix proteins (ECM), epidermal growth factor-growth factor family (EGF), transforming growth factor alpha or beta (TGF alpha, TGF beta), hepatocyte growth factor (HGF/SF), heparin-binding epidermal growth factor (EGF), basic fibroblast growth factor (bFGF), acidic fibroblast growth factor (aFGF), other fibroblast growth factors (FGF), keratinocyte growth factor (KGF), transforming growth factors (TGF) (e.g., beta-1, beta-2, and beta-3), platelet derived growth factor (PDGF), vascular endothelial growth factors (VEGF), tumor necrosis factor (TNF), interleukins, interleukin-1 (IL-1), interleukin-6 (IL-6), other interleukin/cytokine family members, insulin-like growth factor 1 (IGF-1), colony-stimulating factor 1 (CSF-1), and granulocyte macrophage colony stimulating factor (GM-CSF). Additionally, the bioactive substance can be an ion, such as copper, or ion complex, such as a copper amino acid chelate or copper sulfate, that can enhance vascularization. Also, the bioactive substance can be selenium, selenium salt, selenium amino acid chelate, cobalt, cobalt salt, cobalt amino acid chelate, platelet rich plasma (PRP), mammalian cell, a transformed mammalian cell configured to produce a bioactive substance, or the like. The present invention is not limited to the listed angiogenic agents, and additional bioactive substances that are determined to promote vascularization can be included in the bioceramic endoprosthesis.

Accordingly, proteins, peptides, polypeptides, drugs, cytokines, ECM components, ECM mimicking components, and nucleic acids encoding for a bioactive polypeptide, such as an angiogenic factor, may be configured for inkjet printing into the bioceramic. Also, the bioactive substance can be suitable to accelerate healing, induce blood vessel formation, determine tissue type formed, prevent scar/fibrous tissue formation, improve cell attachment, pattern or direct cell migration, prevent infection, transfect surrounding tissue, and the like. Also, cells that can provide or promote a biological function can be bioactive substances and can be included in the bioceramic as described herein. Examples of cells are those that can induce tissue repair, cell migration, cell proliferation, cell or tissue differentiation, would healing, tissue growth, induce vascularization within and locally surrounding the endoprosthesis, and the like.

In the instance the bioactive substance is mammalian cell or transformed mammalian cell, the cells can be characterized by at least one of the following: being combined with a hydrogel carrier; being a cell in a heterogenous population of cells types combined with the endoprosthesis; having platelet rich plasma (PRP); being a cell in a population of autologous cells combined with the endoprosthesis; being a cell in a population of allogeneic cells combined with the endoprosthesis; have been lethally irradiated; or have been treated exogenously with a growth factor.

Additionally, some of the foregoing bioactive substances are temperature sensitive and susceptible to degradation or denaturation under common processing techniques. As such, a “temperature sensitive bioactive substance” is meant to refer to a bioactive substance that can degrade or denature at elevated temperatures. While any substance or compound may be capable of degrading at elevated temperatures, “temperature sensitive bioactive substance” is specifically intended to refer to bioactive substances that are susceptible to degradation or denaturation at elevated temperatures, such as temperatures that degrade or denature proteins or polypeptides. However, a “temperature sensitive bioactive substance” is intended to cover any bioactive substance and should not be construed to be limited to proteins or polypeptides. In any event, the process of preparing the bioceramic can be performed at a low temperature such that the temperature sensitive bioactive substance does not degrade or denature. This can include a low temperature that is lower than a temperature that renders the bioactive substance biologically inactive. Partial degradation may be allowable as long as at least a portion of the bioactive substance is biologically functional. Further, the printing, spraying, or deposition steps that occur with a bioactive substance or after the bioactive substance has been incorporated into the bioceramic can be performed at the low temperature.

Those skilled in the art will recognize that additional bioactive molecules or substance can also be used in the methods and compositions of the invention. Examples of other types of bioactive substance can include drugs that promote healing, inhibit infection, reduce pain, and the like.

Also, a ceramic or cement powder may contain polymeric powders, granules, microspheres, and the like that can be incorporated into the bioceramic. Alternatively, the polymer can be formulated into a solution so as to be capable of being inkjet printed into the bioceramic. The polymer can be biodegradable or biostable. A biodegradable polymer can be useful for incorporation into the bioceramics so that a pore or cavity forms when the polymer degrades. Also, biodegradable polymers can be useful for controlling release of a bioactive substance by bioerosion. A biostable polymer can be useful for incorporation into the ceramic as to form non-erodible or stable features, and may also be useful for controlling release of the bioactive substance by controlling diffusion through the biostable polymer. In some instances, the polymer can function as a binder for the ceramic powder, or function to modulate the physical and/or mechanical properties of the bioceramic. In other instances, the polymer can be a carrier for the bioactive substance, such as in a microsphere or by being co-printed with the bioactive substance. Also, the polymer can be configured to be directly inkjet printed into the bioceramic as is commonly performed in rapid prototyping inkjet systems and methods.

Polymers that do not substantially inhibit the cement setting reaction may be included in the ceramic powder or printed into the bioceramic in order to change diffusion characteristics of the cement matrix for the bioactive substance. For example, gelatin, collagen, or chitosan can be added in regions where plasmid DNA (e.g., encoding for a polypeptide bioactive substance), DNA/carrier, or DNA/carrier/microsphere is added. Also, polymers that are binders or adhesives can be used to bind the ceramic powder, which can include (polyacrylates, polysiloxanes, polyisobutylenes) and the like.

In one embodiment, the bioceramic can be impregnated with a polymer in order to control release of the bioactive substance from spatially localized depots within the endoprosthesis. Examples of such polymers include hydrogels, alginates, polysaccharides, hyaluronic acids, or the like. Also, the polymers can be configured such that they form a hydrogel with calcium during the fabrication process. Also, such polymers, such as hydrogels, can be incorporated into or onto the endoprosthesis as described with respect to the bioactive substance. That is, the polymer, such as a hydrogel, can be present in discrete locations, homogeneously distributed, or heterogeneously distributed.

Additionally, some of the bioactive substance and/or polymer can be incorporated into the bioceramic post printing. This can include depositing, absorbing, or otherwise impregnating the bioactive substance and/or polymer into the bioceramic matrix. For example, a second bioactive substance can be absorbed into the ceramic matrix after printing the first bioactive substance and/or hydrating the cementious composition.

In one embodiment, the bioceramic endoprosthesis includes a biocompatible ceramic matrix having a body defining the external surface of the endoprosthesis, and a bioactive substance being spatially localized within the endoprosthesis. The bioactive substance can be spatially localized within the endoprosthesis by at least one of the following: spatially localized within the ceramic matrix; disposed on a surface of the ceramic matrix, which can be a surface of a pore or external surface; disposed in the ceramic matrix; disposed within a depot; spatially localized in three-dimensions within the endoprosthesis; spatially localized in a two-dimensional pattern within the endoprosthesis; spatially localized in at least one ring or layer; spatially localized in at least two concentric rings or layers; and the like. As described, the bioactive substance disposed in a ring or layer that is three-dimensional, a layer of bioactive substance within the endoprosthesis; disposed at a closed end of at least one pore; or the like.

In one embodiment, the bioceramic includes a diffusion matrix containing a depot of bioactive substance. Optionally, the diffusion matrix is comprised of a polymer. In another option, the diffusion matrix is comprised of at least one pore. In yet another option, a portion of the endoprosthesis can be biodegradable.

In one embodiment, the bioceramic endoprosthesis includes at least one pore. Usually, the pore has an opening in the external surface. Also, the pore can be a part of a network of pores, wherein a portion of the network of pores can be interconnected. As such, the bioactive substance is capable of diffusing into the pore. Additionally, the bioactive substance is capable of diffusing out of the endoprosthesis.

II. Manufacturing Bioceramic Endoprosthesis

Additionally, embodiments of the present invention can include a system and process to prepare the bioceramic having the bioactive substance, such as an angiogenic growth factor, in order to form the bioceramic endoprosthesis. As such, the present invention can include a direct rapid prototyping system and process to manufacture the bioceramic endoprosthesis so as to include the bioactive substance. This can include a system and process that employs inkjet printing technologies in the direct rapid prototyping system and process to co-print the ceramic and bioactive substance to form the bioceramic endoprosthesis. The system and process can be configured such that a temperature sensitive bioactive substance, such as VEGF, can be incorporated into the bioceramic matrix without substantial degradation or denaturation. Such systems and processes are described in more detail below.

In one embodiment, the system and process for preparing the bioceramic endoprosthesis can include direct three-dimensional (3D) printing. Direct 3D powder printing can be used for rapid prototyping or for large-scale manufacturing. As such, the bioceramic can be custom made or prepared in an assembly line manner. Rapid prototyping commonly refers to a class of technologies that can automatically construct physical models in 3D from Computer-Aided Design (CAD) files. Rapid prototyping machines can be considered to be three dimensional printers that allow for prototypes or functional products to be quickly created and manufactured. In addition to prototypes, rapid prototyping systems and processes can also be used to make production-quality objects and is sometimes referred to as rapid manufacturing. For small production runs and complicated objects, rapid prototyping can be advantageous over other manufacturing processes. This is especially true given that the systems and processes can be modulated to account for various temperatures, pressures, or other processing limitations that may be imposed by a particular product or reagent (e.g., temperature sensitive bioactive substance). While the process is relatively fast, some bioceramics may require from three to seventy-two hours to build, depending on the size and complexity of the endoprosthesis.

In order to design a bioceramic endoprosthesis, a software package virtually-slices a CAD model into a number of thin (about 100 microns) layers so that the direct inkjet printing component can then built up one layer atop another in order to form the endoprosthesis. As such, direct inkjet printing is an additive process that combines successive layers of ceramic and/or bioactive substance to create a solid endoprosthesis. Generally, direct inkjet printing can include the following steps: create a CAD model of the design using a computing system; convert the CAD model to STL format or other appropriate format using the computing system; slice the STL file into virtual thin cross-sectional layers using the computing system; physically construct the model one layer atop another layer by sequentially inkjet printing each layer in successive steps; and clean and finish the bioceramic endoprosthesis.

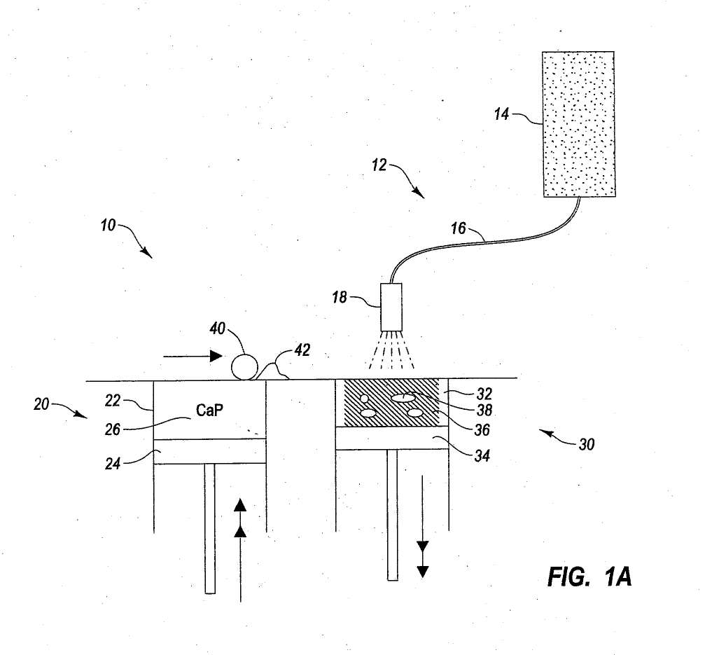

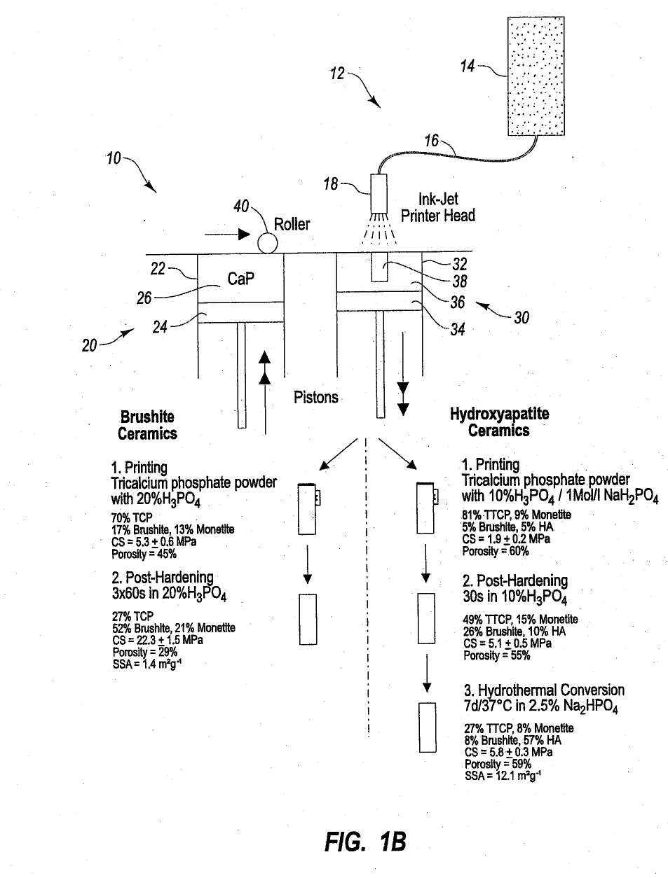

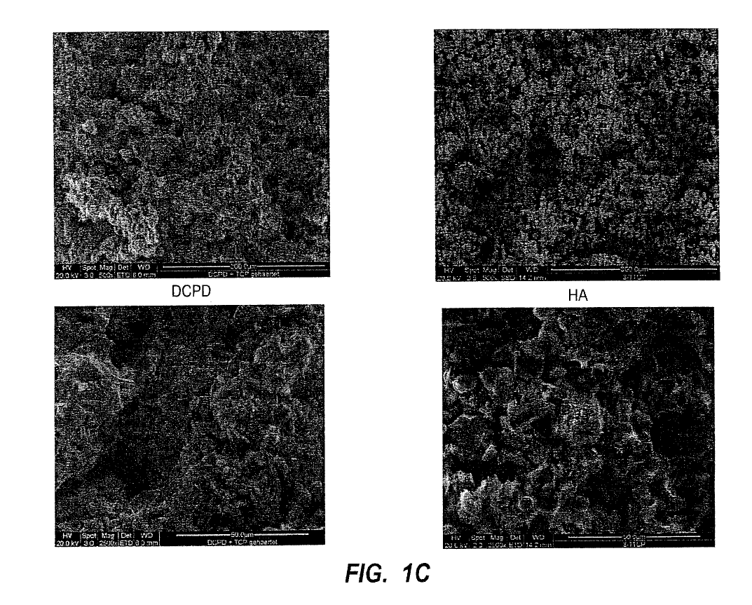

FIGS. 1A-1B illustrate embodiments of a direct rapid prototyping inkjet printing system and process for using inkjet technologies in order to prepare a bioceramic having a bioactive substance. Generally, direct rapid prototyping inkjet systems and methods that are well known in the art can be configured to operate under the present invention. Briefly, such direct rapid prototyping systems can be configured to operate at room temperature or under minimal heat so that proteins or polypeptides can be included within the printing cartridges, reservoirs, and/or ceramic matrix without undergoing degradation or denaturation. As such, direct rapid prototyping systems and processes can be configured to eliminate a sintering step or other step that causes excessive heat and/or pressure. As used herein, direct inkjet printing refers to an entire class of machines that employ inkjet technology to sequentially build a bioceramic endoprosthesis layer-by-layer. An example of such a direct inkjet printer capable of operating under the present invention is a ZCorp 3D printer, produced by Z Corporation of Burlington, Mass.

FIG. 1A depicts an embodiment of a direct inkjet printing system 10 and process in accordance with the present invention. The direct inkjet printing system 10 includes an inkjet printer 12, a powder delivery system 20, a roller 40, and a fabrication system 30.

The inkjet printer 12 has at least one inkjet cartridge 14 that can include any composition capable of being inkjet printed. Additionally, the inkjet printer 12 includes an inkjet line 16 that routes the inkjet composition from the inkjet reservoir 14 to an inkjet printer head 18. Also, the inkjet printer 12 can be configured to include any number of cartridges 14, lines 16, or printer heads 18. Usually, the inkjet printer 12 includes at least one binder cartridge and at least one bioactive substance cartridge.

The powder delivery system 20 has at least one powder delivery chamber 22 that provides a chamber for a powder delivery piston 24. In combination, the powder delivery chamber 22 and powder delivery piston 24 cooperate to contain the ceramic powder 26. The powder delivery piston 24 is configured to move upward as shown by the arrows after each layer of powder is used in the direct inkjet printing process.

The roller 40 is depicted to be a conventional rolling object, such as one rolling part of a calender, which can roll a layer 42 of the ceramic powder 26 from the powder delivery system 20 to the fabrication system 30. However, a squeegee or other similar mechanical instrument can be used to scrape or move a top layer of ceramic powder from the powder delivery system 20 to the fabrication system 30.

The fabrication system 30 has at least one fabrication chamber 32 that provides a chamber for a fabrication piston 34. In combination, the fabrication chamber 32 and fabrication piston 34 cooperate to contain the bioceramic endoprosthesis 36 as it is being fabricated. The fabrication piston 24 is configured to move downward as shown by the arrows after each layer of powder is deposited onto the endoprosthesis 36 and fixed by a binder solution contained in an inkjet cartridge 14.

As shown, the bioceramic endoprosthesis 36 is built in the fabrication chamber 32 on a substrate or platform situated on or integral with the fabrication piston 34. As such, the powder delivery piston 24 rises so that a top layer 42 of the ceramic powder 26 in the powder delivery chamber 22 is rolled by the roller 40 into the fabrication chamber 32. After the lop layer 42 of the ceramic powder 26 is deposited onto the fabrication piston 34, the inkjet printing head 18 selectively deposits or inkjet prints a binder fluid to cure or otherwise fuse the ceramic powder 26 together in the desired areas. Unbound powder can remain to support the part or bound layer of the bioceramic endoprosthesis that has been hardened. After hardening the bound layer, the fabrication piston 34 is lowered, more ceramic powder 26 is added from the powder delivery chamber 22 to the fabrication chamber 34 and leveled, and the process is repeated. Typical layer thicknesses are on the order of 100 microns. When finished, the bioceramic endoprosthesis is considered to be a green body that is then removed from the unbound ceramic powder, and excess unbound powder is blown off or washed away.

At some point in the process, which can be before, during, or after the ceramic is hardened, the bioactive substance can be inkjet printed or otherwise deposited onto the bound powder. The inkjet printing of the bioactive substance can be performed so as to form a bioactive substance reservoir 38 within the bioceramic endoprosthesis 36. While a bioactive substance reservoir 38 is described herein, such a reservoir can be a single molecule, substance, or cell or a depot having a plurality of molecules, substances, or cells. In any event, the green body is prepared so as to have the bioactive substance. This can be used to provide discretely located bioactive substances, to provide bioactive substance gradients, or to homogeneously distribute the bioactive substance throughout the bioceramic matrix. Alternatively, the bioactive substance can be contained in microspheres that are mixed into the ceramic powder 26 and applied therewith.

The printed body having the bioactive substance can then be cured or otherwise finished into a bioceramic endoprosthesis. While the printed body can be partially cured or hardened during printing, an additional curing step can be advantageous to finish the product. Such curing or finishing can be performed at low temperatures by immersing the printed body into a curing solution or hardening solution that causes the ceramic powder to react and harden to its fully hardened state. However, other low temperature curing techniques can be employed that retain the functionality and integrity of the bioactive substance.

In one embodiment, the direct inkjet printing process starts by depositing a layer of ceramic powder 26 at the top of a fabrication chamber 32. To accomplish this, a measured quantity of ceramic powder 26 is first dispensed from a similar supply chamber 22 by moving a piston 24 upward incrementally. The roller 40 then distributes the ceramic powder 42, and compresses the powder at the top of the fabrication chamber 32. A multi-channel inkjet printing head 18 subsequently deposits a liquid binder in a two dimensional pattern onto the layer of the powder which becomes bonded in the areas where the binder is deposited to form a layer of the bioceramic endoprosthesis (bound powder). The multi-channel inkjet printing head 18 subsequently deposits a liquid composition having a bioactive substance in a two dimensional pattern onto the layer of the bound powder to form a bioactive substance reservoir 38. The bioactive substance composition can be configured to be retained on the bound powder by also comprising various additives, binders, gels, pastes, or adhesives. Also, the bioactive substance composition can be configured to dry on the bound powder, absorbed or incorporated during the binding stage or binding reaction, or configured to be retained as a liquid after a subsequent powder layer is applied and bound on top of the bioactive substance. Once a layer is completed, the fabrication piston 34 moves down by the thickness of a layer.

In one embodiment, the bioactive substance is co-printed with the binder so that the bioactive substance is incorporated within the bioceramic matrix. This can include the bioactive substance and binder being intermittently inkjet printed in one pass of the printing head, or the bioactive substance and binder can be inkjet printed in different passes of the printing head, or deposited in any manner during 3D printing of the bioceramic. However, the binder and bioactive substance can be printed with different printing heads or even different printing devices.

In one embodiment, the process of depositing powder and inkjet printing binder is repeated until a substantial portion of the endoprosthesis is formed within the powder bed so as to have an open chamber of unbound powder. The endoprosthesis is then elevated and the unbound powder within the open chamber is then blown or washed away to leave an open chamber. Any system and/or process that can be used to remove unbound powder can be included within the present invention. The bioactive substance can then be inkjet printed into the open chamber, and subsequent layers of powder can be deposited and bound over the chamber so as to form a bioactive substance reservoir 38. The open chamber can be as small as one layer or can be as large as multiple layers of bound powder.

In one embodiment, a spraying process or other deposition process can be used to impregnate the bioceramic with the bioactive substance. As such, the inkjet system and process can include any fluid deposition device to deposit the bioactive substance within the bioceramic matrix.

Additionally, the methods of the present invention can be performed with a spraying device other than an inkjet printer. As such, the recitation herein of direct inkjet printing can also include direct spraying. Accordingly, systems and methods that can be used for spraying compositions can be adapted to for use in the system and methods described herein.

Furthermore, the process of preparing the bioceramic can be performed at a low temperature such that the temperature sensitive bioactive substance does not degrade or denature. This can include a low temperature that is lower than a temperature that degrades or denatures the bioactive substance. Also, this can include temperatures lower than sintering temperatures, which are temperatures required to sinter the ceramics of the present invention. Such temperatures can be less than about 1000° C., less than about 800° C., less than about 600° C., less than about 400° C., less than about 200° C., less than about 100° C., less than about 50° C., or less than about 37° C., depending on the bioactive substance. Further, the printing or spraying steps that occur with a bioactive substance or after some of the bioactive substance has been incorporated into the bioceramic can be performed at the low temperature.

Additionally, multiple materials can be incorporated into the direct printing process described herein, as well as other methods of making the bioceramic endoprosthesis. This can include using two materials simultaneously or nearly simultaneously in preparing two regions. For example, this can allow for preparing the endoprosthesis to include a hydrogel within the ceramic matrix.

FIG. 1B illustrates a more specific embodiment of the direct inkjet printing system and process described in connection with FIG. 1A. As shown, the direct inkjet printing process can be configured for preparing brushite or hydroxyapatite ceramics. The inkjet printing system is configured such that the inkjet cartridge includes a phosphoric acid solution that can be used to bind the ceramic powder. However, other compositions that can bind the ceramic powder may be used.

The process for preparing the brushite ceramic includes the direct inkjet printing process described herein. As shown, the ceramic powder includes about 70% TCP, 17% brushite, and about 13% monetite, and the binder solution is a 20% phosphoric acid solution that can cause the ceramic to harden so as to be capable of being handled without substantial deformation or breakage. The printed ceramic was tested to have a compressive strength (CS) of about 5.3 MPa and to have a porosity of about 45%. In order to increase the mechanical properties, the printed ceramic is then processed in a post-hardening process by immersing or soaking the printed ceramic into a hardening solution. As shown, the hardening solution is a 20% phosphoric acid solution, and the ceramic is hardened by being immersed in the hardening solution 3 times for 60 seconds. The hardening solution should be maintained at a low temperature as described herein. After hardening, the ceramic was characterized as having about 27% TCP, 52% brushite, and about 21% monetite, and having a CS of about 22.3 MPa, porosity of about 29%, and specific surface area (SSA) of about 1.4 m²/g.

The process for preparing the hydroxyapatite ceramic includes the direct inkjet printing process described herein. As shown, the ceramic powder includes about 81% TTCP, 5% brushite, about 5% hydroxyapatite, and about 9% monetite. The binder solution is a 10% phosphoric acid and 1 Mol NaH₂PO₄ solution that can cause the ceramic to harden so as to be capable of being handled without substantial deformation or breakage. The printed ceramic was tested to have a CS of about 1.9 MPa and to have a porosity of about 60%. In order to increase the mechanical properties, the printed ceramic is then processed in a post-hardening process by immersing or soaking the printed ceramic into a hardening solution. As shown, the hardening solution is a 10% phosphoric acid solution, and the ceramic is hardened by being immersed in the hardening solution for 30 seconds. After hardening, the ceramic was characterized as having about 49% TTCP, 26% brushite, 10% hydroxyapatite, and about 15% monetite. The ceramic also had a CS of about 5.1 MPa and porosity of about 55%. The hardened ceramic is then processed in a hydrothermal-conversion process by being immersed or soaked into an aqueous solution for 7 days at 37° C. While hydrothermal-conversion was conducted at 37° C., other low temperatures, such as those described herein, can be used. As shown, the aqueous solution can include 2.5% NaH₂PO₄. After hydrothermal-conversion, the ceramic was characterized has having about 27% TTCP, 8% brushite, 57% hydroxyapatite, and about 8% monetite, and having a CS of about 5.8 MPa, porosity of about 59%, and SSA of about 12.1 m²/g.

However, the post-hardening process and/or hydrothermal-conversion process can be avoided in some instances. As such, the binder solution composition or amount inkjet printed into the ceramic powder can be modified in order to provide for hardening or hydro-conversion. Also, the duration that the bound powder is allowed to harden or cure can be increased before each successive layer is fabricated. In any event, the direct inkjet printing process described herein can be performed so as to retain the functionality of the bioactive substance incorporated into the bioceramic.

Generally, direct 3D printing can be performed by the following process: depositing a first layer of ceramic powder, such as ceramic, at the top of a fabrication chamber or on a substrate; inkjet printing a bonding agent or curing agent onto the first layer of the ceramic powder; bonding or curing the first layer of ceramic powder into a first bioceramic layer; inkjet printing a first layer of a bioactive substance composition on a portion of the first bioceramic layer to form a first bioactive substance layer; depositing a subsequent layer of ceramic powder on the first bioceramic layer and the first bioactive substance layer; inject printing the bonding agent or curing agent onto the subsequent layer of ceramic powder; and bonding or curing the subsequent layer of ceramic powder to form a subsequent bioceramic layer.

In one embodiment, the present invention includes a direct inkjet printing method for preparing a bioceramic endoprosthesis having a releasable bioactive substance at a low temperature. Such a direct inkjet printing method includes the following: (i) applying a ceramic powder to a substrate; (ii) inkjet printing a binder or reactive solution onto the ceramic powder so as to form a bound ceramic; (iii) inkjet printing a bioactive substance solution onto the bound ceramic, wherein the bioactive substance is printed on the bound ceramic at the low temperature; and (iv) repeating steps (i-ii). Optionally, step (iii) can be performed intermittently or concurrently with step (ii). Also, the method can be performed at a low temperature, at room temperature, or within +/−10° C. of 25° C., within +/−20° C. of 25° C., or even within +/−30° C. of 25° C.

In one embodiment, the a direct inkjet printing method can include applying a hardening solution to the bound ceramic, and hardening the bound ceramic into a hardened ceramic having the releasable bioactive substance. In some instances, the direct inkjet printing method can further include applying an aqueous solution to the hardened ceramic maintaining a hydrothermal-conversion or aqueous-conversion temperature of the hardened ceramic while in contact with the aqueous solution so as to further harden the hardened ceramic. Usually, the hydrothermal-conversion temperature is higher than the low temperature. For example, the hydrothermal-conversion temperature can be performed at a low temperature, at room temperature, or within +/−10° C. of 37° C., within +/−20° C. of 37° C., or even within +/−30° C. of 37° C., or lower than a temperature that degrades or denatures the bioactive substance.

In one embodiment, the direct inkjet printing method can include fabricating the bioceramic endoprosthesis so as to have at least one pore having a diameter greater than about 200 microns; and localizing a portion of the bioactive substance within a ceramic matrix adjacent to a surface of the pore. This can include the bioactive substance being inkjet printed into a reservoir adjacent to the surface of the pore or printed into the bioceramic matrix adjacent the pore.

In one embodiment, the bioceramic material can be hardened, bound, or cured in a process similar to the hardening or curing of a cementitious material. The hardening, binding, or curing may occur by the reaction of the ceramic powder on contact with a liquid. This process may occur by any of the known calcium phosphate cement forming reactions, or the like. For example, this process may occur through the reaction of an appropriate hardening, binding, and/or curing composition with any of the following: calcium phosphate; calcium oxide; hydroxide phase; mixture of phases; mechanically activated compound; amorphous compound; glass compounds; or the like. Examples of hardening, binding, and/or curing composition include any of the following: water; solutions of phosphate; solutions of pyrophosphate; solutions of polyphosphate; solutions of carbonate; solutions of silicate; solutions of phosphonate; solutions of alpha hydroxyacid; solutions of sulphate ions; solutions of acids; aqueous solutions there; and mixtures thereof.

Examples of ceramic powders and hardening, binding, and/or curing composition pair include the following: tetracalcium phosphate is reacted with phosphoric acid solution; beta tricalcium phosphate is reacted with phosphoric acid solution; beta tricalcium phosphate is reacted with pyrophosphoric acid solution; beta tricalcium phosphate is reacted with polyphosphoric acid solution; alpha tricalcium phosphate is reacted with phosphoric acid and/or sodium phosphate solution; tetracalcium phosphate, dicalcium phosphate (dihydrate and or anhydrous), and/or mixtures thereof are reacted with sodium phosphate solutions.

Additionally, a process substantially similar to FIGS. 1A-1B can be employed using a hydrogel or polymeric composition. Such a process can then incorporate the hydrogel or polymer into the endoprosthesis as described in connection with the bioactive substance. Moreover, the hydrogel or polymer can be included into the endoprosthesis with the bioactive substance.

The bioceramic endoprosthesis of the present invention can be prepared by other methods. As such, the bioceramic endoprosthesis can be made by casting the ceramic in a mold and then adding the bioactive substance. Such molding can include molds that provide the pores, channels, or other features described herein. Also, holes can be drilled into the endprosthesis to provide the pores, channels, or other features described herein. Also, the ceramic endoprosthesis can be prepared by machining a block into the shape of the endoprosthesis, which can optionally include forming the pores, channels, or other features. Examples of methods for preparing the ceramic endoprosthesis body can be found in U.S. Pat. No. 6,905,516, which is incorporated herein by specific reference. After the ceramic endoprosthesis is prepared or during low temperature processing, the bioactive substance can be added to the endoprosthesis as described herein. Thus, the ceramic endoprosthesis can be prepared by rapid prototyping, molding, machining, sintering, and/or compacting.

III. Characterization Of Endoprosthesis

It has been shown that grafts taken from the patient's own skeleton (autograft) induce angiogenesis by active endogenous signalling molecules, a property lacking in allograft and synthetic grafts.^{[10]} Formation of a blood supply is an important initial step in the growth of new tissues; it not only nourishes cells involved with healing but also provides a source of osteoprogenitor cells. Inducing angiogenesis in synthetic porous grafts using VEGF has been shown to accelerate bone healing,^{[11,12]} which is important because there is a limited supply of spare bone for autografting in the body and graft harvesting requires an additional surgical procedure. Prior concerns over disease transmission from donor allogenic bone have been heightened recently,^{[13]} further reinforcing the need for improvements in bone graft substitute bioceramics. Regenerative products based on tissue induction following protein release from polymeric matrices are now a reality in the field of bone and periodontal surgery.^{[14,15]} However, protein mediated tissue regeneration is not without drawbacks, which include cost of production, supply and storage of an unstable recombinant product, and perceived risks of delivering higher than physiological levels of potent inductive factors.

As described herein, a direct rapid prototyping inkjet printing process was used to prepare 3D powder print bioceramic structures at room temperature. Direct 3D inkjet printing at room temperature is highly significant because it allows simultaneous control of geometry of the bioceramic and control of bioactive substance (e.g., organic molecule) incorporation in the bioceramic. The direct 3D inkjet printing of the bioceramic can allow for replication of biomimetic micro-environments for controlled tissue healing by having a bioceramic endoprosthesis that releases the bioactive substance. Furthermore, the utility of the direct 3D inkjet printing process was demonstrated by directly fabricating model bioceramic implants from brushite (i.e., dicalcium phosphate dihydrate), hydrated calcium phosphate, and hydroxyapatite so as to include organic angiogenic factors, such as vascular endothelial growth factor (VEGF). As such, bioactive substances that are susceptible to degradation under high temperatures can now be included in a bioceramic due to the low temperature processing. In any event, the following discussion of experiments and results relates to experiments that were conducted in accordance with the Experimental Protocols section provided below.

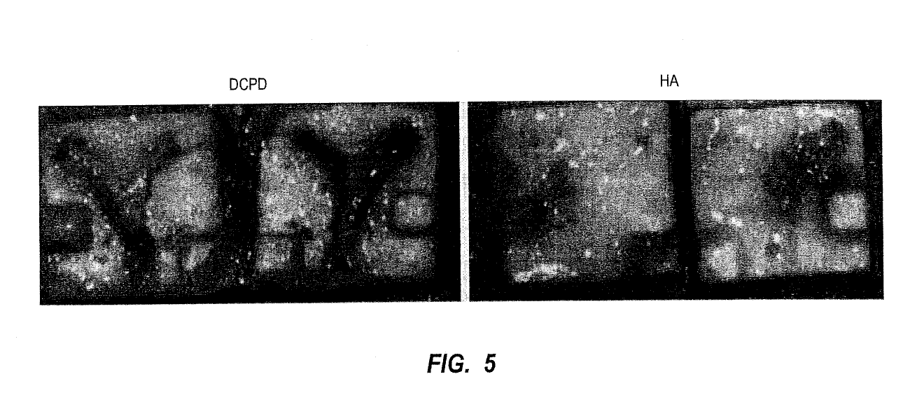

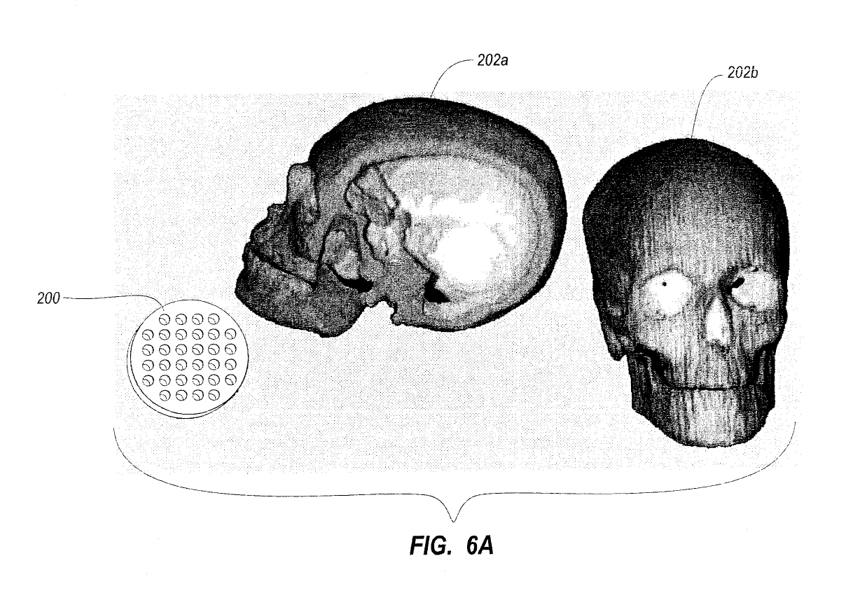

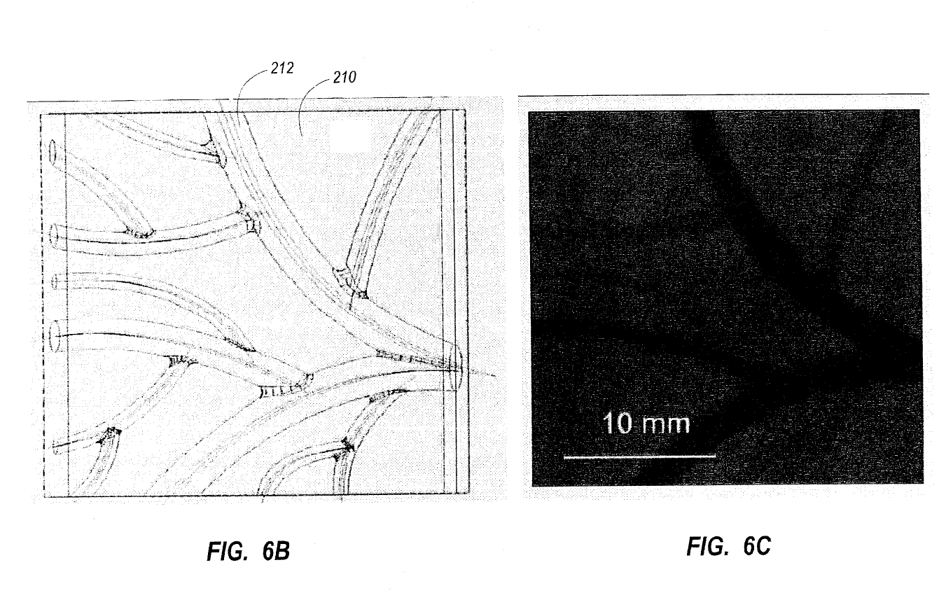

As described in more detail above with respect to FIGS. 1A-1B, fabrication of the bioceramic endoprosthesis was performed using a direct rapid prototyping 3D inkjet printing technique in a two step process. Either brushite (i.e., dicalcium phosphate dihydrate—DCPD) or hydroxyapatite (HA) can be made into complex shapes by using tricalcium (TCP) or tetracalcium phosphate (TTCP) powders respectively (see FIG. 6A). Each layer is printed to be about 100 μm thick; however, the thickness can be modulated as needed or desired. The 100 μm thick layers took approximately 8-12 seconds to print depending on the print area.

FIGS. 6A-6C shows that programmed complex shapes could be replicated from scaled down CT data or from hand drawn computer aided design (CAD) files. As shown, the methods of the present invention can provide a variety of 3D shapes. This can include a disk 200 having holes, a skull 202a,b, a block 210 having channels 212, and others.

The mean compressive strengths of DCPD and HA components directly after printing were 5.3±0.6 and 1.9±0.2 MPa, respectively. This was an important finding as the post-printed cements were strong enough to be handled without breaking or fragmenting during removal of the unreacted powders using compressed air. After post-print hardening or aqueous conversion, compressive strength increased 3-4 fold to about 22.3±1.5 MPa for DCPD and about 5.8±0.3 MPa for HA. This increase in strength was caused by an increase in the degree of conversion of powder reactants from 30% to 73% for DCPD and from 19% to 57% for HA samples (FIG. 1B) and these strengths are higher than values reported for a commercial sintered bone graft substitute.^{[9]} Post-printing hardening and aqueous conversion hardly affected the microporosity and pore size of HA samples. The porosity decreased from 60% to 59%, and the median pore size decreased from 15 microns to 12 micron. In contrast, the porosity of the DCPD samples decreased from 46% to 29%, and the median pore size decreased from 27 microns to 13 microns after post-printing hardening.

FIG. 1C provides micrographs that show the set cements to include large (10-20 μm) angular particles of unreacted starting powder in a matrix of tabular crystals of DCPD (5-10 μm) or platy crystals 2-5 μm of HA.

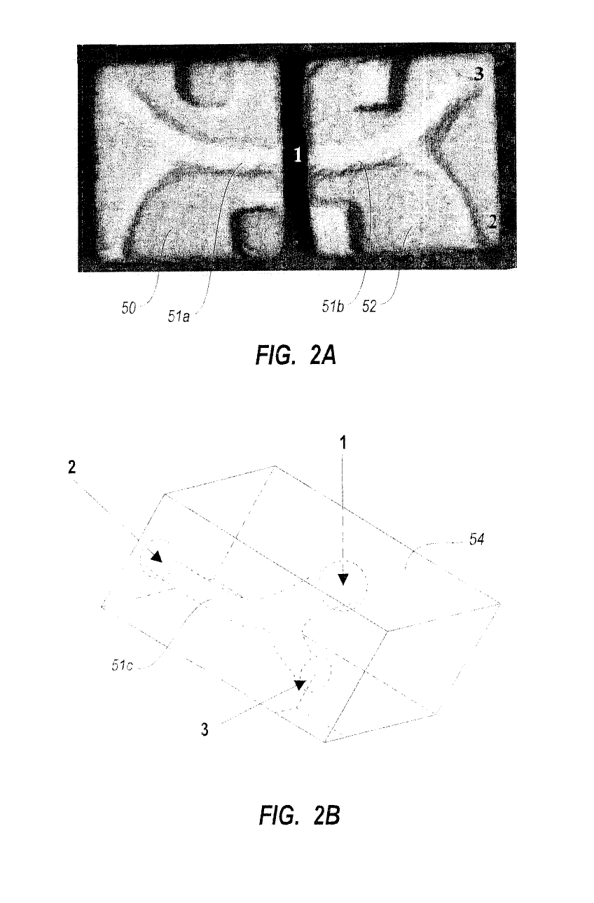

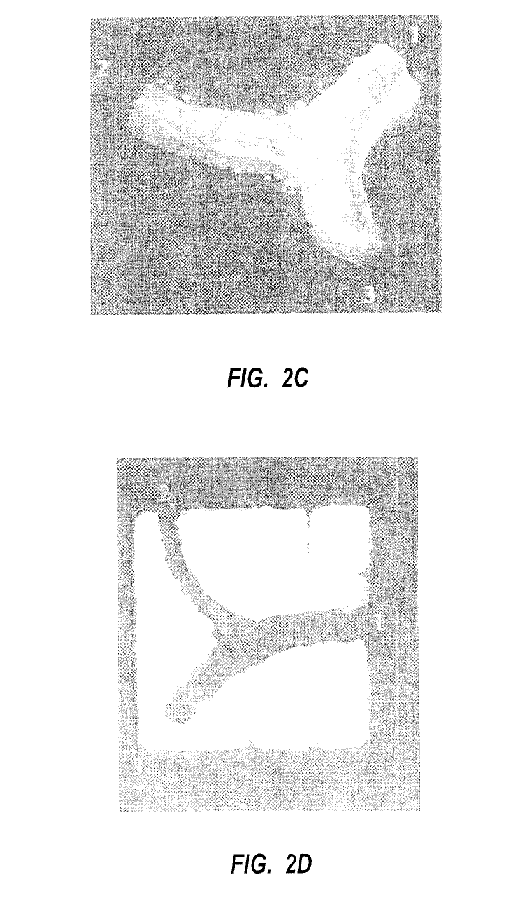

FIGS. 2A-2D depict a process of producing a model using computing technologies and direct rapid prototyping inkjet printing in accordance with the present invention. In order to produce model implants for the investigation of spatially localized release of both organic (VEGF), a Y shaped hemi-cylindrical pore channel was designed in the x-y plane of DCPD cuboids with dimensions 8 mm×8 mm×3 mm. The bioceramic endoprosthesis was made in two mirror image halves 50, 52 that keyed into one another to form a Y shaped pore 51a, 51b closed at one of the branched ends (see FIGS. 2A-2C). This design facilitated tissue examination. Micro-computed tomography (μCT) revealed the block 54 and pore 51c architecture (FIGS. 2B-2D) and demonstrated that the main open pore had a diameter of 1.31±0.11 mm (FIG. 2D). In the experiments, mouse VEGF solution was deposited at the end of the closed pore.

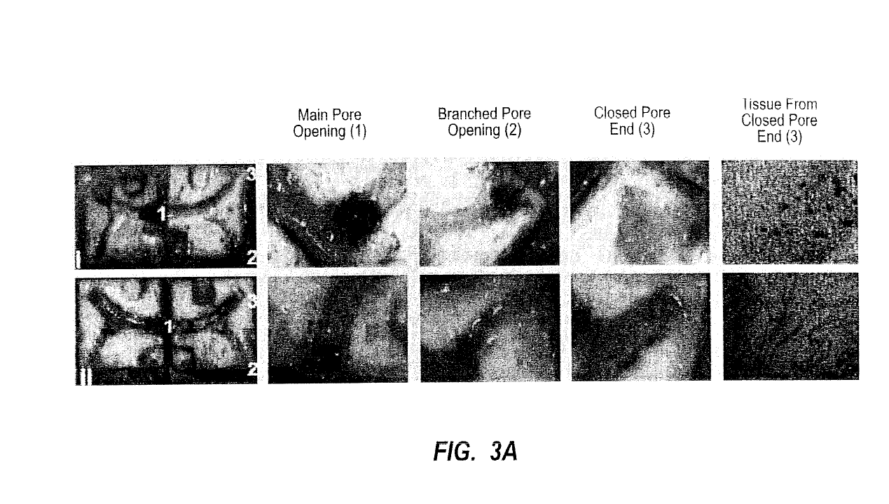

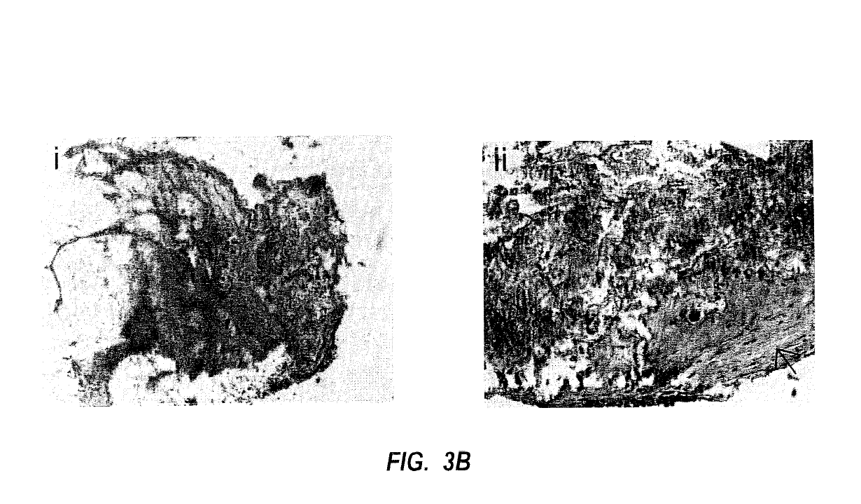

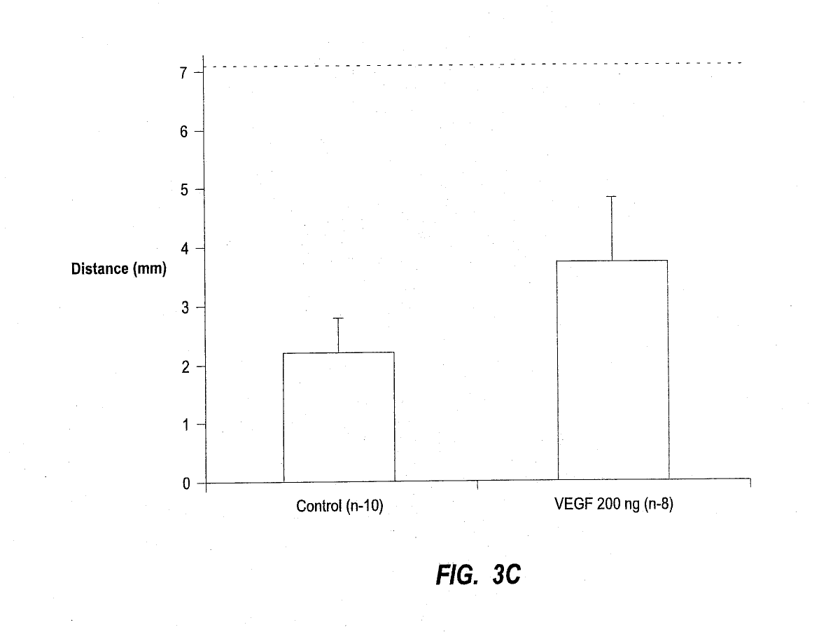

Experiments using the bioceramic endoprosthesis showed that after peritoneal implantation for 15 days, blood vessels or microvessels had only entered 2 mm into the open pores of the factor-free model implants (controls), while blood vessels extended the entire length of the pore (7 mm) towards the regions where the angiogenic factors had been deposited (see FIG. 3A). Histology of the tissue confirmed the presence of an organized microvessel network in the experimental implants (see FIG. 3B (i-ii)). Quantitative measurements confirmed that VEGF elicited a significantly enhanced angiogenic effect compared with an untreated DCPD control (see FIG. 3B).

Wound tissue and angiogenesis patterns were examined for the implanted bioceramic endoprosthesis after peritoneal implantation of the bioceramic endoprosthesis in mice for 15 days. After opening the mating halves which remained tightly sealed throughout implantation, tissue response was found to be both material and angiogenic factor dose dependant. DCPD untreated control implants only had limited tissue ingrowth at the pore openings. DCPD with 200 ng and 2 μg VEGF deposited in pore ends had vascular tissue throughout the pore channels, conversely within the pores of HA implants containing VEGF very little tissue ingrowth was apparent (FIG. 3B (ii)). These observations may have been a result of the differences in specific surface area between HA and DCPD implants (see FIG. 1C), or differences in their solubility. Based on these observations DCPD was selected as a matrix and optimal loading levels of 200 ng VEGF were used for subsequent implantation to quantify microvessel ingrowth.

Vascularised tissue was found inside the main open and closed pore channels of the DCPD implants which had been loaded with 200 ng VEGF (FIG. 3A) localized at the closed pore ends.

It was found that 3D protein concentration gradients could be achieved by repeated application of decreasing volumes of serum protein solution. Stability of these treatments was confirmed in water and in serum for up to 3 weeks. Thus we demonstrated that localized and controlled protein and ion binding could be achieved that would initially remain stable post implantation.

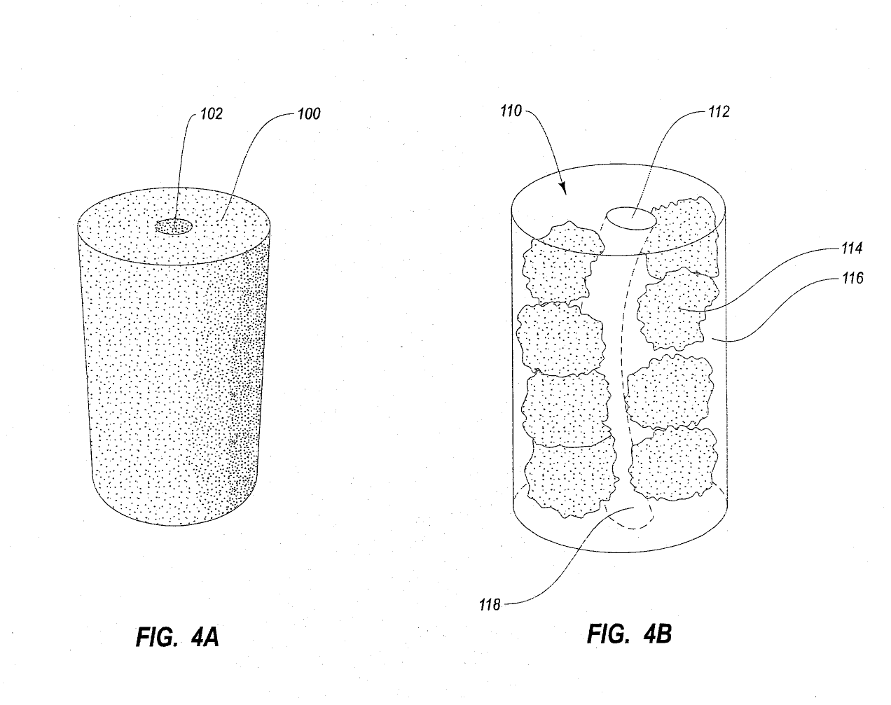

FIG. 4A illustrates an embodiment of a bioceramic endoprosthesis 100 having a channel 102 extending therethrough. The endoprosthesis 100 having the channel 102 was prepared as described herein. FIG. 4B is a schematic representation of a μCT image of a bioceramic endoprosthesis 110 having a channel 112 extending therethrough. Additionally, the endoprosthesis 110 includes depots 114 of a bioactive substance within the ceramic matrix 116. While not required, the endoprosthesis 110 is shown to have a sealed end 118 so as to have a sealed channel 112 at one end.

To demonstrate the feasibility of controlling both 3D architecture and composition of the bioceramic endoprosthesis as part of the powder printing process, 150 mM barium chloride solution was deposited in a pattern of eight 40 μl spots during printing of a cylinder with a curved central pore. As shown in FIG. 4B, μCT revealed that the barium chloride remained localized where it had been applied during cement setting. Additionally, the post-print hardening process can be optimized by either allowing full setting to occur post-implantation or in more physiological conditions, such as by optimizing the powder physics of a two powder component cement system. In one example, supplying a source of reactive phosphate ions in the powder phase or binder may obviate the use of acidic phosphate solutions for curing the bioceramic.

The direct rapid prototyping inkjet printing process, as described herein, may be used to prepare bone graft endoprostheses that are fabricated from CT or MRI 3D data files with tissue inductive protein, peptide, or other biologically bioactive substances applied locally to generate a regenerative response by more closely mimicking the complex natural tissue regeneration process. In part, the local concentration of the bioactive substance within or adjacent to the implanted bioceramic endoprosthesis can mimic or induce the tissue regeneration process. As such, it is conceivable that this direct rapid prototyping inkjet printing process can provide a bioceramic having reservoirs or depositions of bioactive substance that can replicate biological responses that have been observed to be induced by bone autograft. Indeed, recent preclinical reports have indicated that bone formation was enhanced by the use of VEGF loaded hydrogels combined with biomineral grafts.^{[21]}

The methods of the present invention can be used to prepare anatomical endoprostheses (i.e., implants), such as orthopedic implants that can be implanted into the head, neck, torso, arms, legs, and the like. For example, the methods can be used to prepare cranial implants.

Although this work was performed with a single inkjet printer, multi-head inkjet printers, which can include multiple reservoirs of different fluids (e.g., multiple binders, bioactive substances, polymers, or the like) can be utilized in the powder printing systems of the direct rapid prototyping inkjet printing process described herein, or separate depositions systems. Thus, using the direct rapid prototyping inkjet printing process reported here, it is possible to print multiple compositions into the bioceramic. For example, a 5 head inkjet printer could be configured as follows: the first inkjet cartridge solution can include the binding agent; the second inkjet cartridge solution can include a angiogenic agent; the third inkjet cartridge solution can include a gene delivery system that encodes an angiogenic protein; the fourth inkjet cartridge solution can include an antibiotic; and the fifth inkjet cartridge can include a pain reliever. However, it should be realized that multiple different binding agents could be employed or any other variation that employs a multi-head inkjet printer. This could provide for the systematic study of multiple bone and vascularized tissue factors and for development of improved patient-tailored bone graft substitutes. Alternatively, other systems and/or processes that can spray one or more binders, bioactive substances, or polymers can also be used.

FIG. 5 are photographs depicting vascularization patterns in pores of DCPD (left) and HA (right) implants with 2 μg VEGF deposited in closed pore ends opened following 15 days implantation. HA implants were mostly filled with clear connective tissue.

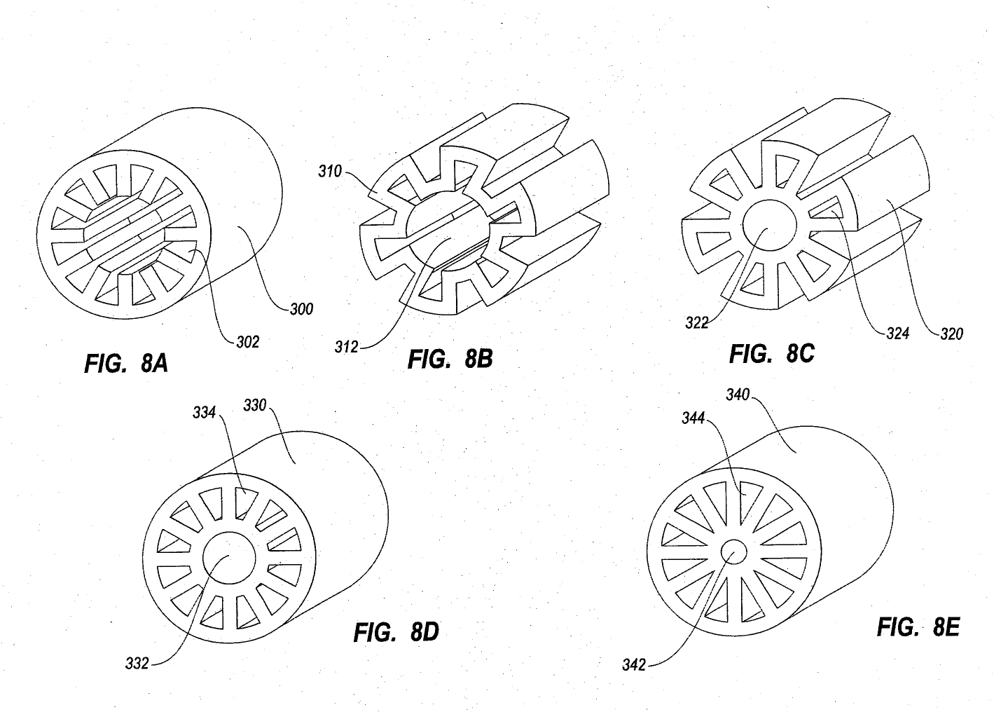

FIGS. 8A-8E are schematic representations of embodiments of endoprostheses of the present invention. FIG. 8A illustrates a tubular endoprosthesis 300 having a corrugated internal lumen 302. FIG. 8B illustrates a scalloped endoprosthesis 310 having a corrugated internal lumen 312. FIG. 8C illustrates a scalloped endoprosthesis 320 having a primary lumen 322 and a plurality of channels 324 that longitudinally extend through the endoprosthesis 320. The primary lumen 322 and plurality of channels 324 are independent and non-intersecting. FIG. 8D illustrates a tubular endoprosthesis 330 having a primary lumen 332 and a plurality of channels 334 that longitudinally extend through the endoprosthesis 320. The primary lumen 332 and plurality of channels 334 are independent and non-intersecting. FIG. 8E illustrates a spoke-shaped tubular endoprosthesis 340 having a primary lumen 342 and a plurality of spoke channels 344 that longitudinally extend through the endoprosthesis 340. The primary lumen 342 and plurality of spoke channels 344 are independent and non-intersecting.

Accordingly, the present invention can be directed to methods of making a bioceramic endoprosthesis and the product thereof.

In on embodiment, the present invention can include a direct printing method for preparing a bioceramic endoprosthesis having a bioactive substance. Such a method can include: (i) applying a ceramic powder to a substrate; (ii) spraying a binder solution onto the ceramic powder so as to form a bound ceramic; (iii) depositing at least one bioactive substance solution onto the bound ceramic so as to incorporate the bioactive substance with bound ceramic; and (iv) repeating steps (i-ii) or (i-iii).

In one embodiment, the depositing of the bioactive substance and step (iii) is performed at a low temperature that is lower than a temperature that degrades or denatures the bioactive substance. Optionally, the low temperature is room temperature or within +/−10° C. of 25° C.

In one embodiment, the method includes applying a hardening solution to the bound ceramic, and hardening the bound ceramic into a hardened ceramic having the releasable bioactive substance.

In one embodiment, the method includes applying an aqueous solution to the hardened ceramic, and maintaining a hydrothermal-conversion temperature of the hardened ceramic while in contact with the aqueous solution so as to further harden the hardened ceramic, wherein the hydrothermal-conversion temperature is higher than the low temperature.

In one embodiment, the hydrothermal-conversion temperature is +/−10° C. of 37° C. Also, the hydrothermal-conversion temperature can be lower than a temperature that degrades or denatures the bioactive substance.

In one embodiment, the entire method of preparing the endoprosthesis is performed at a temperature that is lower than a temperature that degrades or denatures the bioactive substance.

In one embodiment, at least one of the binder or bioactive substance is inkjet printed. Optionally, the bioactive substance can be sprayed into a reservoir within a ceramic matrix of the bioceramic endoprosthesis. Also, the bioactive substance can be sprayed so as to form a concentration gradient within a ceramic matrix of the bioceramic endoprosthesis. In another option, the bioactive substance can be sprayed so as to be substantially homogeneously distributed throughout at least a portion of a ceramic matrix of the bioceramic endoprosthesis.

In one embodiment, the method of preparing the endoprosthesis includes fabricating the bioceramic endoprosthesis so as to have at least one pore having a diameter greater than about 200 microns, and localizing a portion of the bioactive substance within a ceramic matrix adjacent to a surface of the pore.

In one embodiment, the method includes absorbing a bioactive substance into the bioceramic matrix.

In one embodiment, the present invention includes a system for low temperature printing a bioceramic endoprosthesis having a releasable bioactive substance. Such a system can include an inkjet printer comprising: at least one inkjet reservoir containing a binder composition capable of being inkjet printed; at least one inkjet reservoir containing a bioactive substance composition capable of being inkjet printed; and at least one an inkjet printer head capable of inkjet printing the binder composition and/or the bioactive substance composition.

In one embodiment, the system includes a powder delivery system comprising: at least one powder delivery chamber containing a ceramic powder; and a powder delivery piston configured to raise the ceramic powder layer-by-layer.

In one embodiment, the system includes a fabrication system comprising: at least one fabrication chamber configured for receiving the ceramic powder, and for receiving the binder composition and bioactive substance from the at least one inkjet printer head so as to form a bound ceramic having the bioactive substance; and a fabrication piston configured to lower the bound ceramic having the bioactive substance layer-by-layer.

In one embodiment, the system includes a roller configured to distribute a top layer of the ceramic powder from the powder delivery system to the fabrication system.

In one embodiment, the present invention includes a bioceramic endoprosthesis. Such a bioceramic endoprosthesis can include a biocompatible ceramic matrix having a body defining the external surface of the endoprosthesis, and a bioactive substance being spatially localized within the endoprosthesis. Optionally, the bioactive substance is spatially localized within the ceramic matrix. In another option, the bioactive substance is disposed on a surface of the ceramic matrix. For example, the surface is a surface of a pore. In another example, the bioactive substance is disposed in the ceramic matrix. In yet another example, the bioactive substance is disposed within a depot.

In one embodiment, the endoprosthesis includes a diffusion matrix containing the depot. Such a diffusion matrix can include a polymer.

In one embodiment, the bioceramic endoprosthesis includes at least one pore or a network of interconnected or non-connected pores. For example, the bioactive substance is disposed at a closed end of the at least one pore. In another example, the pore has an opening in the external surface.

In one embodiment, the bioactive substance is spatially localized in three-dimensions within the endoprosthesis. Alternatively, the bioactive substance is spatially localized in a two-dimensional pattern within the endoprosthesis. In another embodiment, the bioactive substance is spatially localized in at least one ring or layer. In yet another embodiment, the bioactive substance is spatially localized in at least two concentric rings or layers. For example, the ring or layer is three-dimensional. This can include the ring or layer forming a ring or layer of bioactive substance within the endoprosthesis.

In one embodiment, at least a portion of the endoprosthesis is biodegradable.

In one embodiment, the bioactive substance stimulates tissue growth within and/or around the endoprosthesis.

In one embodiment, the bioactive substance is capable of diffusing into the pore.

In one embodiment, the bioactive substance is capable of diffusing out of the endoprosthesis.

### EXPERIMENTAL PROTOCOLS

### Example 1

TTCP was synthesized by heating an equimolar mixture of dicalcium phosphate anhydrous (DCPA, CaHPO₄, monetite) (Merck, Darmstadt, Germany) and calcium carbonate (CC, CaCO₃, calcite) (Merck, Darmstadt, Germany) to 1500° C. for 18 hr followed by quenching to room temperature. The sintered cake was crushed with a pestle and mortar and passed through a 160 μm sieve. Milling was performed in a planetary ball mill (PM400, Retsch, Germany) at 200 rpm with 500 ml agate jars, 4 agate balls with a diameter of 30 mm and a load of 125 g TTCP per jar for 30 min. 55 wt % β/45 wt % α TCP was prepared in a similar manner by sintering a 2:1 molar mixture of DCPA and CC at 1400° C. for 14 h.

### Example 2

Printing of cement samples was performed with a 3D-powder printing system (Z-Corporation, USA) using the TTCP or TCP powder and as liquid printing phases either a mixture of 10% phosphoric acid and 1M NaH₂PO₄ (both Merck, Darmstadt, Germany) for TTCP powder or 20% phosphoric acid solution for TCP powder. Sample geometries were designed using CAD software or directly taken from CT scan data. Inkjet printing was performed using an anisotropic scaling with x=y=z=1.0. Samples printed from TTCP were additionally stored in 10% H₃PO₄ for 30 s followed by immersion in 2.5% Na₂HPO₄ solution at 37° C. for 7 days in order to convert to HA. TCP printed samples were stored in 20% H₃PO₄ for 60 s 3 times to increase the degree of reaction to DCPD. More detailed information regarding the direct rapid prototyping inkjet printing process of the present invention can be reviewed above and in connection with FIGS. 1A-1C.

### Example 3

Compressive strength testing, as reported above (see FIG. 1B), was performed 24 h after setting with cylindrical samples (10>n>5) with a height of 20 mm and a diameter of 10 mm under axial compression at a crosshead speed of 1 mm/min using a static mechanical testing device (Zwick 1440, Ulm, Germany) and a 5 kN load cell. The porosity of the printed samples was calculated from the apparent density after drying and the strut density from the phase composition and using literature density values.^{[29]} Pore size distributions were measured by high pressure Hg-porosimetry (Porosimeter 2000, Carlo Erba Instr., Milano, Italy). The Brunnauer Emmet Teller method (BET) was used to determine the specific surface area within the porous calcium phosphate matrices (Micromeritics, ASAP 2000, USA). The microstructure of gold sputtered fracture surfaces was characterized by scanning electron microscopy (FEI, Quanta 200, Czech Republic). X-ray micrographs of porous structures were taken with a Polydoros SX80 (Siemens, Germany) at 40 KV and 5.0 mAs. Additional information regarding the experimental protocols can be found in the incorporated references, or is well known to those of ordinary skill in the art.

### Example 4

X-ray diffraction (XRD) patterns of samples were recorded using monochromatic Cu K_{α} radiation (D5005, Siemens, Karlsruhe, Germany). Data was collected from 2θ=20-40° with a step size of 0.02° and a normalized count time of 1 s/step. The phase composition was checked by means of ICDD reference patterns for α-TCP (PDF Ref. 09-0348), β-TCP (PDF Ref. 09-0169), DCPA (PDF Ref. 09-0080), HA (PDF Ref. 09-0432), and DCPD (PDF Ref. 09-0077). Quantitative phase relations of the composite materials were calculated by means of total Rietveld refinement analysis using the TOPAS software AXS, Karlsruhe, Germany). As references the system internal database structures of α-TCP, β-TCP, HA, DCPD and DCPA were used together with a Chebychev fourth order background model and a Cu K_{α} emission profile.

### Example 5

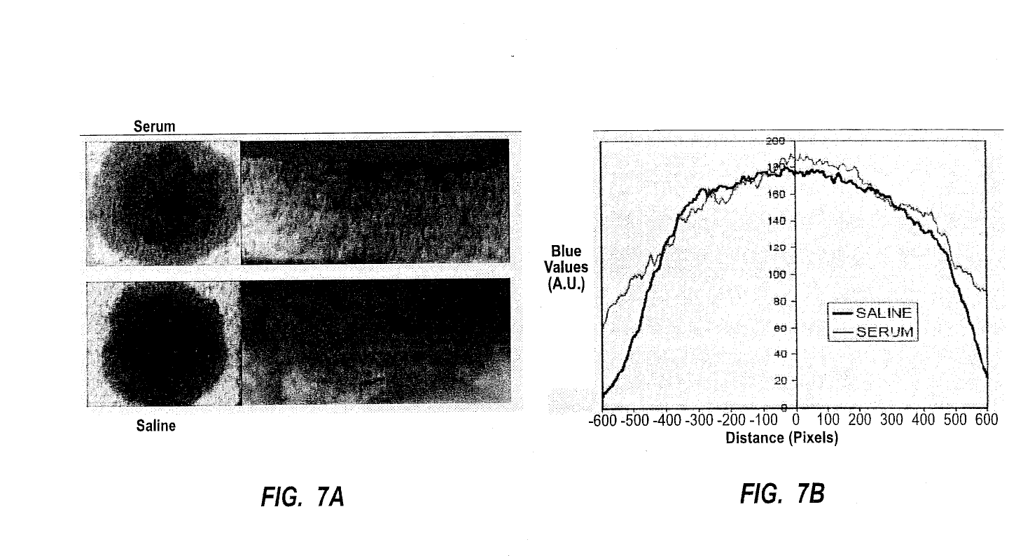

Serum protein concentration gradients, in response to bioceramics including VEGF, were determined with respect to the bioceramic (FIG. 7). Briefly, 1.8 mg/ml bovine serum protein or 0.45% saline Coomassie blue solutions (0.05% final) were applied in decreasing volumes to surfaces of DCPD and HA blocks (e.g., 5, 2.5, 1 and 0.5 μl) to create stepped gradients of concentration. Coomassie blue binding to protein was determined to be 90% efficient (by ultra-centrifugation). Images were recorded with a Q-Imaging video camera and analysis was performed with Q-Capture software (Quantitative Imaging Corporation). The blue value as a function of distance was measured diametrically across the stained region of the cements (Image J, Scion Corporation). Serum protein concentration was measured by spectrofluorometry at 595 nm (Protein assay Bioraid, Biorad). FIG. 7 shows the gradients of serum proteins and saline on DCPD were, stable in vitro for up to 3 weeks.

### Example 6

For experimental analysis, VEGF-impregnated bioceramic materials were prepared by passive adsorption of 200 ng or 2 μg final amount of mouse VEGF (R&D, Cedarlane Laboratories Ltd., Canada) diluted in a 5 μl volume of Hank's balanced salt solution (HBSS). Adsorption of VEGF was performed on each half of the bioceramic endoprosthesis (see FIGS. 2A-2D) at the closed end (3) of the Y-shape pore. Specimens were air-dried under a sterile laminar flow hood. Prior to VEGF impregnation, the bioceramic materials were sterilized by soaking in 70% ethanol, followed by a HEPES-NaCl buffered solution rinse, HBSS (×3), and then air-dried. Control material specimens were also impregnated with HBSS without VEGF. Higher concentrations of VEGF (400 μg/ml) were also investigated.

### Example 7

Micro-computed tomography was performed on a Skyscan Model 1072, (Aartselaar, Belgium). The x-ray source was operated at 70 kV and at 142 μA (maximum power). Images were captured using a 12-bit, cooled CCD camera (1024 by 1024 pixels). Samples were scanned at a magnification resulting in a pixel size of 14.08 μm. A rotation step of 0.68° and an exposure time of 9.2 s for each step with a 0.5 mm aluminum filter were used. The cross-sections along the specimen axis were reconstructed using NRec Reconstruction software (SkyScan) giving a voxel size of 14.08×14.08×14.08 μm³. 3D Creator software (SkyScan) was used to perform 3D rendering.

### Example 8

Flat square demonstration samples (50×50×5 mm) with colored stripes on the surface with an increasing wideness of 1-5 mm were printed with a multicolor 3D printing machine Z510 (Z-Corporation, Burlington, Mass., USA) using tricalcium phosphate with a medium particle size of approximately 30 μm as powder and 20% diluted phosphoric acid as binder solution in the binder reservoir. Printing parameters were a layer thickness of 125 μm and a binder to powder volume ratio of approx. 0.3. A 5% copper sulfate solution was used in a second reservoir of the printer and sprayed with printing head 1 (which is assigned to the yellow color information of the “.wrl” file) to obtain stripes of deposited copper sulfate on the top surface of the printed calcium phosphate sample.”

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope. All references recited herein are incorporated herein in their entirety by specific reference.

The following references provide background information regarding features of the present invention, and are incorporated herein by specific reference in their entirety.
[1] P. Habibovic et al., Biomaterials, 2005, 26, 3565.
[2] V. Karageorgiou, D. Kaplan, Biomaterials 2005, 26, 5474.
[3] D. Kochan, C. K. Chua and Z. H. Du, Computers in Industry: an International Journal, 1999, 39, 3.
[4] W. Y. Yeong, C. K. Chua, K. F. Leong and M Chandrasekaran, Trends in Biotechnology 2004, 22, 643.
[5] C. K. Chua, K. F. Leong, K. H. Tan, F. E. Wiria, C. M. Cheah, J. Mater. Sci.: Mater. Med. 2004, 15, 1113.
[6] H. Seitz, W. Rieder, S. Irsen, B. Leukers, C. Tille, J. Biomed. Mater. Res. 2005, 74, 782.
[7] Z. Sadeghian, J. G. Heinrich, F. Mortaradeh, CFI-Ceramic Forum International, 2004, 81, E39.
[8] C. E. Wilson, J. D. de Bruijn, C. A. van Blitterswijk, A. J. Verbout, W. J. A. Dhert, J. Biomed. Mater. Res. 2004, 68, 123.
[9] L. Xiang et al., Rapid Prototyp. J. 2005, 11, 312.
[10] K. A. Hing, S. M. Best, K. E. Tanner, W Bonfield, P. A. Revell J. Mater. Sci.-MatS. in Med. 1997, 8 731.
[11] H. Ito et al., Nat. Med. 2005, 11, 291
[12] H. Eckardt M. Ding, M. Lind, E. S. Hansen, K. S. Christensen, I. Hvid, J. Bone Joint Surg. Br. 2005, 87, 1434.
[13] FDA Public Health Notification: Update of Information about BioMedical Tissue Services, Mar. 2, 2006 http://www.fda.gov/cber/safety/bts030206.htm
[14] M. Boakye, P. V. Mummaneni, M. Garrett, G. Rodts, R. Haid, J. Neurosurg: Spine 2, 521-525 (2005).
[15] A. R. Vaccaro, T. Patel, J. Fischgrund, D. G. Anderson, E. Truumees, H. N. Herkowitz, F. Phillips, A. Hilibrand, T. J. Albert, T. Wetzel, J. A. McCulloch, Spine 29, 1885-1892 (2004).
[16] G. Hu, J. Cell. Biochem. 1998, 6, 326.
[17] A. Parke, P. Bhattacherjee, R. M. Palmer, Am. J. Pathol. 1988, 130, 173.
[18] C. K. Sen et al., Am. J. Physiol. Heart Circ. Physiol. 2002, 282, H1821.
[19] G. Giavaresi et al., Biomaterials 2005, 26, 3001.
[20] M. Bohner, U. Gbureck, J. E. Barralet, Biomaterials, 2005, 26, 6423.
[21] W. L. Murphy, C. A. Simmons, D. Kaigler, D. J. Mooney, J. Dent. Res. 2004, 83, 204.
[22] M. R. Urist, Science, 1965, 150, 893.
[23] D. E. Sims, Tiss. Cell 1986, 18, 153.
[24] C. T. Brighton, Clin. Orthop. Relat. Res. 1992, 275, 287.
[25] M. J. Doherty et al., J. Bone Min. Res. 1998, 13, 828.
[26] Holzel T., Rapid prototyping of calcium phosphate structures by means of a cement setting reaction. Diploma Thesis, University of Applied Sciences, Erlangen-Nürnberg, Germany, Aug. 19, 2005.

1-23. (canceled)

24. A method for preparing a bioceramic endoprosthesis, the method comprising:
(i) providing a ceramic powder;
(ii) applying a binder solution onto the ceramic powder so as to form a bound ceramic;
(iii) depositing at least one of the following onto the bound ceramic so as to be incorporated with the bound ceramic:
a bioactive substance containing solution at a temperature that does not degrade the bioactive substance;
a hydrogel; or
a polymer; and
(iv) repeating steps (i-ii) or (i-iii).

25. A method as in claim 24, wherein applying the binder solution onto the ceramic powder forms an endoprosthesis.

26. A method as in claim 24, further comprising applying the ceramic powder to a substrate prior to applying the binder solution.

27. A method as in claim 24, wherein the binder is sprayed onto the ceramic powder.

28. A method as in claim 24, further comprising:
defining an anatomically designed bioceramic endoprosthesis based on a patient CT scan or MRI; and
preparing the anatomically designed bioceramic endoprosthesis to have the bioactive substance, hydrogel, or polymer.

29. A method as in claim 24, further comprising:
configuring the bound ceramic to be a multi-layered pill having the bioactive substance.

30. A method as in claim 24, wherein the method for forming the ceramic endoprosthesis includes at least one of the following: rapid prototyping; molding; machining; or compacting.

31. A method as in claim 24, further comprising:
applying a hardening solution to the ceramic; and
hardening the ceramic into a hardened ceramic having the bioactive substance, hydrogel, or polymer.

32. A method as in claim 31, further comprising:
applying an aqueous solution to the hardened ceramic; and
maintaining a hydrothermal-conversion temperature of the hardened ceramic while in contact with the aqueous solution so as to further harden the hardened ceramic.

33. A method as in claim 24, wherein one or more of the bioactive substance, hydrogel, or polymer is:
not homogeneously distributed in the endoprosthesis; or
deposited in discrete and selected locations of the endoprosthesis.

34. A method as in claim 24, wherein the bioactive substance is selected from the group consisting of extracellular matrix component, synthetic extracellular matrix component, proteins, peptides, polypeptides, drugs, cytokines, DNA, RNA, cells, bone-inducing factors, bone morphogenic proteins (BMPs), growth factors, extra cellular matrix proteins (ECM), epidermal growth factor-growth factor family (EGF), transforming growth factor alpha or beta (TGF alpha, TGF beta), hepatocyte growth factor (HGF/SF), heparin-binding epidermal growth factor (EGF), basic fibroblast growth factor (bFGF), acidic fibroblast growth factor (aFGF), other fibroblast growth factors (FGF), keratinocyte growth factor (KGF), transforming growth factors (TGF), TGF beta-1, TGF beta-2, TGF beta-3, platelet derived growth factor (PDGF), vascular endothelial growth factors (VEGF), tumor necrosis factor (TNF), interleukin-1 (IL-1), interleukin-6 (IL-6), other interleukin/cytokine family members, insulin-like growth factor 1 (IGF-1), colony-stimulating factor 1 (CSF-1), and granulocyte macrophage colony stimulating factor (GM-CSF), copper, copper salt, copper amino acid chelate, copper sulfate, selenium, selenium salt, selenium amino acid chelate, cobalt, cobalt salt, cobalt amino acid chelate, platelet rich plasma (PRP), mammalian cell, a transformed mammalian cell, bacteria cell, transformed bacteria cell configured to produce a bioactive substance, and combinations thereof.

35. A method as in claim 24, further comprising:
combining a mammalian cell or a transformed mammalian cell configured to produce a bioactive substance with the endoprosthesis, said mammalian cell or transformed mammalian cell being characterized by at least one of the following:
being combined with a hydrogel carrier; being a cell in a heterogenous population of cells types combined with the endoprosthesis; having platelet rich plasma (PRP);
being a cell in a population of autologous cells combined with the endoprosthesis;
being a cell in a population of allogeneic cells combined with the endoprosthesis have been lethally irradiated; or
have been treated exogenously with a growth factor.

36. A method as in claim 24, wherein the ceramic powder is selected from the group consisting of bioinert ceramic, alumina, surface-bioactive ceramics, silicon carbide, zirconia, hydroxyapatite (HA), bioglasses, resorbable bioactive ceramics, alpha and/or beta tricalcium phosphates (TCP), tetracalcium phosphate (TTCP), octacalcium phosphate, calcium sulfate, dicalcium phosphate dihydrate (DCPD), hydrated calcium phosphates, calcium hydrogen phosphate, dicalcium phosphate anhydrous (DCPA), low-crystallinity HA, calcium pyrophosphates (anhydrous or hydrated), calcium polyphosphates (n>3), calcium polyphosphate, calcium silicates, calcium carbonate, amorphous calcium salts, whitlockite, zeolite, artificial apatite, brushite, calcite, gypsum, phosphate calcium ore, iron oxides, calcium sulphate, magnesium phosphate, calcium deficient apatites, amorphous calcium phosphates, combinations thereof, crystalline forms thereof, amorphous forms thereof, anhydrous forms thereof, or hydrated forms thereof.

37. A method as in claim 24, further comprising on or more of the following:
(a) fabricating the bioceramic endoprosthesis so as to have at least one pore having a diameter greater than about 200 microns, and localizing a portion of the bioactive substance within a ceramic matrix adjacent to or on a surface of the pore;
(b) fabricating the bioceramic endoprosthesis so as to have at least one longitudinal channel, pore, wedge, groove, slot, corrugation, or spoke extending through the endoprosthesis so as to direct tissue growth therethrough; or
combining a pharmacologic excipient with the endoprosthesis, wherein the excipient is selected from fibrin, fibrin sheets, and cell stabilizing composites.

38. A bioceramic endoprosthesis comprising:
a biocompatible ceramic matrix having a body defining a external surface of the endoprosthesis; and
at least one of a bioactive substance, hydrogel, or polymer within the ceramic matrix,
said bioceramic endoprosthesis being prepared by the following:
(i) providing a ceramic powder;
(ii) applying a binder solution onto the ceramic powder so as to form a bound ceramic;
(iii) depositing at least one of the following onto the bound ceramic so as to be incorporated with the bound ceramic:
a bioactive substance containing solution at a temperature that does not degrade the bioactive substance;
a hydrogel; or
a polymer; and
(iv) repeating steps (i-ii) or (i-iii).

39. A bioceramic endoprosthesis as in claim 38, wherein the endoprosthesis is characterized by at least one of the following:
at least one of the bioactive substance, hydrogel, or polymer is spatially localized within the ceramic matrix;
at least one of the bioactive substance, hydrogel, or polymer is disposed on a surface of the ceramic matrix;
the ceramic matrix includes a pore, or pore network of interconnected pores;
the ceramic matrix includes a pore network of non-connected pores; the bioactive substance is spatially localized in at least one ring or layer; at least a portion of the endoprosthesis is biodegradable; the endoprosthesis is configured for oral delivery; the endoprosthesis is configured to be administered orally and pass through the stomach; or
the bioactive substance stimulates tissue growth within and/or around the endoprosthesis.
